SEQUENCE LISTING (29 sequences)

SEQ ID NO: 1 - protein, 77 aa, Homo sapiens
MEVNFAKNRKDKNQTYNLTGLQPFTEYVIALRCAVKESKFWSDWSQEKMGMTEEEGKLLPAIPVLSATGVGLLWARL

SEQ ID NO: 2 - protein, 42 aa, Homo sapiens
MEVTFAKNRKDKNQTYNLTGLQPFTEYVIALRCAVKESKFLE

SEQ ID NO: 3 - DNA, 2480 bp, Homo sapiens
cccacatcttagtgtggataaattaaagtccagattgttcttcctgtcctgacttgtgctgtgggaggtggagttgcctttgatgcaaatcctttgagccagcagaacatctgtggaacatcccctgatacatgaagctctctccccagccttcatgtgttaacctggggatgatgtggacctgggcactgtggatgctcccttcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcatttcagtgcattgaggtcatgcaggcctgcgttgctgaggaccagctagtggtgaagtggcaaagccctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccgacgggaccagcataaatttcaagacattgtcattcagtgtctttgagattatcctcataacttctctgattggtggaggccttcttattctcattatcctgacagtggcatatggtctcaaaaaacccaacaaattgactcatctgtgttggcccaccgttcccaaccctgctgaaagtagtatagccacatggcatggagatgatttcaaggataagctaaacctgaaggagtctgatgactctgtgaacacagaagacaggatcttaaaaccatgttccacccccagtgacaagttggtgattgacaagttggtggtgaactttgggaatgttctgcaagaaattttcacagatgaagccagaacgggtcaggaaaacaatttaggaggggaaaagaatgggtatgtgacctgccccttcaggcctgattgtcccctggggaaaagttttgaggagctcccagtttcacctgagattccgcccggaaaatcccaatacctacgttcgaggatgccagaggggacccgcccagaagccaaagagcagcttctcttttctggtcaaagtttagtaccagatcatctgtgtgaggaaggagccccaaatccatatttgaaaaattcagtgacagccagggaatttcttgtgtctgaaaaacttccagagcacaccaagggagaagtctaaatgcgaccatagcatgagaccctcggggcctcagtgtggatggcccttgccagagaagatgtcaagactcggcacgcagcgcttgcttggccctgccacatcctgcctagg

SEQ ID NO: 4 - protein, 745 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSPALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPGKSQYLRSRMPEGTRPEAKEQLLFSGQSLVPDHLCEEGAPNPYLKNSVTAREFLVSEKLPEHTKGEV

SEQ ID NO: 5 - DNA, 2238 bp, Homo sapiens
atgaagctctctccccagccttcatgtgttaacctggggatgatgtggacctgggcactgtggatgctcccttcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcatttcagtgcattgaggtcatgcaggcctgcgttgctgaggaccagctagtggtgaagtggcaaagccctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccgacgggaccagcataaatttcaagacattgtcattcagtgtctttgagattatcctcataacttctctgattggtggaggccttcttattctcattatcctgacagtggcatatggtctcaaaaaacccaacaaattgactcatctgtgttggcccaccgttcccaaccctgctgaaagtagtatagccacatggcatggagatgatttcaaggataagctaaacctgaaggagtctgatgactctgtgaacacagaagacaggatcttaaaaccatgttccacccccagtgacaagttggtgattgacaagttggtggtgaactttgggaatgttctgcaagaaattttcacagatgaagccagaacgggtcaggaaaacaatttaggaggggaaaagaatgggtatgtgacctgccccttcaggcctgattgtcccctggggaaaagttttgaggagctcccagtttcacctgagattccgcccggaaaatcccaatacctacgttcgaggatgccagaggggacccgcccagaagccaaagagcagcttctcttttctggtcaaagtttagtaccagatcatctgtgtgaggaaggagccccaaatccatatttgaaaaattcagtgacagccagggaatttcttgtgtctgaaaaacttccagagcacaccaagggagaagtctaa

SEQ ID NO: 6 - protein, 1097 aa, Homo sapiens
MMDIYVCLKRPSWMVDNKRMRTASNFQWLLSTFILLYLMNQVNSQKKGAPHDLKCVTNNLQVWNCSWKAPSGTGRGTDYEVCIENRSRSCYQLEKTSIKIPALSHGDYEITINSLHDFGSSTSKFTLNEQNVSLIPDTPEILNLSADFSTSTLYLKWNDRGSVFPHRSNVIWEIKVLRKESMELVKLVTHNTTLNGKDTLHHWSWASDMPLECAIHFVEIRCYIDNLHFSGLEEWSDWSPVKNISWIPDSQTKVFPQDKVILVGSDITFCCVSQEKVLSALIGHTNCPLIHLDGENVAIKIRNISVSASSGTNVVFTTEDNIFGTVIFAGYPPDTPQQLNCETHDLKEIICSWNPGRVTALVGPRATSYTLVESFSGKYVRLKRAEAPTNESYQLLFQMLPNQEIYNFTLNAHNPLGRSQSTILVNITEKVYPHTPTSFKVKDINSTAVKLSWHLPGNFAKINFLCEIEIKKSNSVQEQRNVTIKGVENSSYLVALDKLNPYTLYTFRIRCSTETFWKWSKWSNKKQHLTTEASPSKGPDTWREWSSDGKNLIIYWKPLPINEANGKILSYNVSCSSDEETQSLSEIPDPQHKAEIRLDKNDYIISVVAKNSVGSSPPSKIASMEIPNDDLKIEQVVGMGKGILLTWHYDPNMTCDYVIKWCNSSRSEPCLMDWRKVPSNSTETVIESDEFRPGIRYNFFLYGCRNQGYQLLRSMIGYIEELAPIVAPNFTVEDTSADSILVKWEDIPVEELRGFLRGYLFYFGKGERDTSKMRVLESGRSDIKVKNITDISQKTLRIADLQGKTSYHLVLRAYTDGGVGPEKSMYVVTKENSVGLIIAILIPVAVAVIVGVVTSILCYRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 7 - protein, 979 aa, Homo sapiens
MALFAVFQTTFFLTLLSLRTYQSEVLAERLPLTPVSLKVSTNSTRQSLHLQWTVHNLPYHQELKMVFQIQISRIETSNVIWVGNYSTTVKWNQVLHWSWESELPLECATHFVRIKSLVDDAKFPEPNFWSNWSSWEEVSVQDSTGQDILFVFPKDKLVEEGTNVTICYVSRNIQNNVSCYLEGKQIHGEQLDPHVTAFNLNSVPFIRNKGTNIYCEASQGNVSEGMKGIVLFVSKVLEEPKDFSCETEDFKTLHCTWDPGTDTALGWSKQPSQSYTLFESFSGEKKLCTHKNWCNWQITQDSQETYNFTLIAENYLRKRSVNILFNLTHRVYLMNPFSVNFENVNATNAIMTWKVHSIRNNFTYLCQIELHGEGKMMQYNVSIKVNGEYFLSELEPATEYMARVRCADASHFWKWSEWSGQNFTTLEAAPSEAPDVWRIVSLEPGNHTVTLFWKPLSKLHANGKILFYNVVVENLDKPSSSELHSIPAPANSTKLILDRCSYQICVIANNSVGASPASVIVISADPENKEVEEERIAGTEGGFSLSWKPQPGDVIGYVVDWCDHTQDVLGDFQWKNVGPNTTSTVISTDAFRPGVRYDFRIYGLSTKRIACLLEKKTGYSQELAPSDNPHVLVDTLTSHSFTLSWKDYSTESQPGFIQGYHVYLKSKARQCHPRFEKAVLSDGSECCKYKIDNPEEKALIVDNLKPESFYEFFITPFTSAGEGPSATFTKVTTPDEHSSMLIHILLPMVFCVLLIMVMCYLKSQWIKETCYPDIPDPYKSSILSLIKFKENPHLIIMNVSDCIPDAIEVVSKPEGTKIQFLGTRKSLTETELTKPNYLYLLPTEKNHSGPGPCICFENLTYNQAASDSGSCGHVPVSPKAPSMLGLMTSPENVLKALEKNYMNSLGEIPAGETSLNYVSQLASPMFGDKDSLPTNPVEAPHCSEYKMQMAVSLRLALPPPTENSSLSSITLLDPGEHYC

SEQ ID NO: 8 - protein, 918 aa, Homo sapiens [sequence omitted]

SEQ ID NO: 9 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 10 - protein, 7 aa, Homo sapiens
WKSTSVK

SEQ ID NO: 11 - protein, 15 aa, Homo sapiens
EGKLLPAIPVLSALK

SEQ ID NO: 12 - protein, 726 aa, Mus musculus
MKPLGVNAGIMWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQETILRIPDVHEKTFQYIKSMQAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSISVFEVVLLTSLVGGGLLLLSIKTVTFGLRKPNRLTPLCCPDVPNPAESSLATWLGDGFKKSNMKETGNSGNTEDVVLKPCPVPADLIDKLVVNFENFLEVVLTEEAGKGQASILGGEANEYVTSPSRPDGPPGKSFKEPSILTEVASEDSHSTCSRMADEAYSELARQPSSSCQSPGLSPPREDQAQNPYLKNSVTTREFLVHENIPEHSKGEV

SEQ ID NO: 13 - protein, 252 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEGKLLPAIPVLSTLV

SEQ ID NO: 14 - protein, 652 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMANTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQEKQFRRGKEWD

SEQ ID NO: 15 - protein, 662 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 16 - protein, 344 aa, Homo sapiens [sequence omitted]

SEQ ID NO: 17 - protein, 39 aa, Homo sapiens [sequence omitted]

SEQ ID NO: 18 - protein, 12 aa, Homo sapiens
SNWLALKGDEEK

SEQ ID NO: 19 - DNA, 2830 bp, Homo sapiens
aaagaagacatgacacagccaacaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctggaccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtc

SEQ ID NO: 20 - DNA, 1890 bp, Homo sapiens
atgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtag

SEQ ID NO: 21 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 22 - DNA, 1698 bp, Homo sapiens
atgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtag

SEQ ID NO: 23 - protein, 565 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 24 - DNA, 1071 bp, Homo sapiens
atgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctggattaaaagaaggatcttattgttaa

SEQ ID NO: 25 - protein, 356 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSGLKEGSYC

SEQ ID NO: 26 - protein, 384 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSPFFHKTPETVPQVTSKAFQHDTWNSGLTVASISTGHLTSDNRGDIGLLLGMIVFAVMLSILSLIGIFNRSFPNWD

SEQ ID NO: 27 - protein, 644 aa, Mus musculus
MSHLTLQLHVVIALYVLFRWCHGGITSINCSGDMWVEPGEIFQMGINVSIYCQEALKHCRPRNLYFYKNGFKEEFDITRINRTTARIWYKGFSEPHAYMHCTAECPGHFQETLICGKDISSGHPPDAPSNLTCVIYEYSGNMTCTWNTGKPTYIDTKYIVHVKSLETEEEQQYLASSYVKISTDSLQGSRKYLVWVQAVNSLGMENSQQLHVHLDDIVIPSASIISRAETTNDTVPKTIVYWKSKTMIEKVFCEMRYKTTTNQTWSVKEFDANFTYVQQSEFYLEPDSKYVFQVRCQETGKRNWQPWSSPFVHQTSQETGKRNWQPWSSPFVHQTSQTVSQVTAKSSHEPQKMEMLSATIFRGHPASGNHQDIGLLSGMVFLAIMLPIFSLIGIFNRSLRIGIKRKVLLMIPKWLYEDIPNMENSNVAKLLQEKSVFENDNASEQALYVDPVLTEISEISPLEHKPTDYKEERLTGLLETRDCPLGMLSTSSSVVYIPDLNTGYKPQVSNVPPGGNLFINRDERDPTSLETTDDHFARLKTYPNFQFSASSMALLNKTLILDELCLVLNQGEFNSLDIKNSRQEETSIVLQSDSPSETIPAQTLLSDEFVSCLAIGNEDLPSINSYFPQNVLESHFSRISLFQK

SEQ ID NO: 28 - DNA, 2181 bp, Mus musculus
atgaagcctctgggtgtgaacgctggaataatgtggaccttggcactgtgggcattctctttcctctgcaaattcagcctggcagtcctgccgactaagccagagaacatttcctgcgtcttttacttcgacagaaatctgacttgcacttggagaccagagaaggaaaccaatgataccagctacattgtgactttgacttactcctatggaaaaagcaattatagtgacaatgctacagaggcttcatattcttttccccgttcctgtgcaatgcccccagacatctgcagtgttgaagtacaagctcaaaatggagatggtaaagttaaatctgacatcacatattggcatttaatctccatagcaaaaaccgaaccacctataattttaagtgtgaatccaatttgtaatagaatgttccagatacaatggaaaccgcgtgaaaagactcgtgggtttcctttagtatgcatgcttcggttcagaactgtcaacagtagccgctggacggaagtcaattttgaaaactgtaaacaggtctgcaacctcacaggacttcaggctttcacagaatatgtcctggctctacgattcaggttcaatgactcaagatattggagcaagtggagcaaagaagaaaccagagtgactatggaggaagttccacatgtcctggacctgtggagaattctggaaccagcagacatgaacggagacaggaaggtgcgattgctgtggaagaaggcaagaggagcccccgtcttggagaaaacatttggctaccacatacagtactttgcagagaacagcactaacctcacagagataaacaacatcaccacccagcagtatgaactgcttctgatgagccaggcacactctgtgtccgtgacttcttttaattctcttggcaagtcccaagagaccatcctgaggatcccagatgtccatgagaagaccttccagtacattaagagcatgcaggcctacatagccgagcccctgttggtggtgaactggcaaagctccattcctgcggtggacacttggatagtggagtggctcccagaagctgccatgtcgaagttccctgccctttcctgggaatctgtgtctcaggtcacgaactggaccatcgagcaagataaactaaaacctttcacatgctataatatatcagtgtatccagtgttgggacaccgagttggagagccgtattcaatccaagcttatgccaaagaaggaactccattaaaaggtcctgagaccagggtggagaacatcggtctgaggacagccacgatcacatggaaggagattcctaagagtgctaggaatggatttatcaacaattacactgtattttaccaagctgaaggtggaaaagaactctccaagactgttaactctcatgccctgcagtgtgacctggagtctctgacacgaaggacctcttatactgtttgggtcatggccagcaccagagctggaggtaccaacggggtgagaataaacttcaagacattgtcaatcagtgtgtttgaaattgtccttctaacatctctagttggaggaggccttcttctacttagcatcaaaacagtgacttttggcctcagaaagccaaaccggttgactcccctgtgttgtcctgatgttcccgaccctgctgaaagtagtttagccacatggctcggagatggtttcaagaagtcaaatatgaaggagactggaaactctgggaacacagaagacgtggtcctaaaaccatgtcccgtccccgcggatctcattgacaagctggtagtgaactttgagaattttctggaagtagttttgacagaggaagctgggaagggtcaggcgagcattttgggaggagaagcgaatgagtatgtgacctccccgtctaggcccgacggtcccccagggaaaagttttaaagagccttccattttaactgaggttgcttctgaagactcccacagcacgtgttccagaatggcggacgaggcgtactcagaattggccaggcagccttcgtcttcctgtcagagtccagggctatcgcctccccgtgaagaccaagctcagaatccgtatttgaaaaattcggtgacaaccagggaatttcttgtgcatgagaatgtcccagagcacagcaaaggagaagtctga

SEQ ID NO: 29 - DNA, 1935 bp, Mus musculus
atgagtcacctcacacttcagctgcatgtggtgatagccctttatgtgctcttcagatggtgtcacggaggaatcacaagtataaactgctctggtgacatgtgggttgagcctggtgaaatttttcagatgggcataaatgtttctatatattgccaagaagcccttaagcactgccgaccaaggaatctttacttttataaaaatggcttcaaagaagaatttgatatcacaaggattaatagaacaacagctcggatttggtataaaggcttttcggaacctcatgcctatatgcattgcactgctgaatgtcctggtcattttcaagagacactgatttgtgggaaagacatttcctctggacatccaccggatgcccccagcaatctgacatgtgtcatttatgaatactcaggcaacatgacatgcacctggaacactgggaagcctacctacatagataccaagtatattgtgcatgtgaagagtttggagacagaagaagaacaacaatatcttgcctcaagctatgttaagatctccactgactcactgcaaggcagcaggaagtatttggtatgggtccaagctgtcaattccctaggcatggagaactcacaacaactacacgtccatctggatgatatagtgataccttctgcgtccatcatttccagggctgagactacaaacgatactgtacccaagaccatagtttactggaaaagcaaaactatgattgagaaagtattctgtgagatgagatacaaaacaacaacaaaccaaacgtggagtgttaaagaatttgacgccaatttcacatatgtacagcagtcagaattctacctggagccagacagcaagtatgtatttcaagtgcgatgtcaagaaactggtaaaagaaactggcagccttggagttccccctttgtccaccaaacttcccaagaaactggtaaaagaaactggcagccttggagttcccccttcgtccaccaaacttcccagacagtttcccaggttacagcaaaatcatcccacgaacctcagaagatggagatgctcagtgctacaatcttcagaggacatcctgcttcaggtaatcatcaagacattggacttttgtcgggaatggtcttcttggccatcatgttgccgattttttctctgattgggatatttaacagatcacttcgaataggaattaaaaggaaagttttactgatgatcccaaagtggctttatgaagatattcctaatatggaaaatagcaatgttgcaaaattattacaggaaaaaagtgtatttgagaatgataatgccagtgagcaggccctgtatgtggatcctgtccttacagagataagtgaaatctctcccctggaacacaaacccacagattacaaagaagaaaggctcacaggactccttgagacaagagactgtcctctaggaatgttgtctaccagttcttctgttgtgtatattcctgacctcaacactggatacaaaccccaggtttcaaatgttcctcctggaggaaaccttttcattaacagagatgaaagagaccctacatcccttgagaccacagatgaccactttgccagattgaaaacatatcccaacttccaattttctgcttcaagtatggctttactaaacaaaacactaattcttgatgaattgtgcctcgttttaaatcaaggagaattcaattctcttgacataaaaaactcaagacaggaggaaaccagcatcgttttgcaaagtgactcacccagtgaaactatcccagcgcagactctgttgtctgatgaatttgtctcctgtttggcaattgggaatgaagacttgccatctattaattcttactttccacagaacgttttggaaagccatttcagtagaatttcactcttccaaaagtag

(19) United States
(12) Patent Application Publication
(10) Pub. No.: US 2003/0059871 A1
(43) Pub. Date: Mar. 27, 2003

(54) Hematopoietin receptors HPR1 and HPR2

(76) Inventors: David J. Cosman, Bainbridge Island, WA (US); Bruce A. Mosley, Seattle, WA (US); Timothy A. Bird, Bainbridge Island, WA (US); Robert F. DuBose, Bellevue, WA (US); Steven R. Wiley, Seattle, WA (US)

Correspondence Address: IMMUNEX CORPORATION, LAW DEPARTMENT, 51 UNIVERSITY STREET, SEATTLE, WA, 98101

(21) Appl. No.: 09/972,708

(22) Filed: Oct. 5, 2001

Related U.S. Application Data
(60) Provisional application No. 60/238,706, filed on Oct. 6, 2000.
(60) Provisional application No. 60/240,476, filed on Oct. 13, 2000.
(60) Provisional application No. 60/270,282, filed on Feb. 20, 2001.

(51) Int. Cl. C07K 14/705; C07H 21/04; C12N 5/06; C12P 21/02

(52) U.S. Cl. 435/69.1; 435/325; 435/320.1; 530/350; 536/23.2

(57) ABSTRACT

This invention relates to human and murine HPR1 and human and murine HPR2 polypeptides, new members of the hematopoietin receptor polypeptide family; to methods of making such HPR1 and HPR2 polypeptides; to non-human mammals in which the endogenous genomic sequences encoding HPR1 and/or HPR2 polypeptides have been partially or completely inactivated; to methods of using HPR1 or HPR2 polypeptides to identify compounds that alter HPR1 or HPR2 polypeptide activities; and to methods of preparing medicaments for and/or treating conditions associated with hematopoietin receptor function.

This application claims the benefit under 35 U.S.C. 119(e) of U.S. provisional applications Serial No. 60/238,706, filed Oct. 06, 2000; Serial No. 60/240,476, filed Oct. 13, 2000; and Serial No. 60/270,282, filed Feb. 20, 2001; all of which are incorporated by reference herein.

### FIELD OF THE INVENTION

This invention relates to new human and murine hematopoietin receptor polypeptides HPR1 and HPR2, and to methods of making and using HPR1 and HPR2 polypeptides.

### BACKGROUND OF THE INVENTION

The hematopoietin receptor polypeptides are a related group of Type I membrane protein receptors, and in some cases soluble forms of those receptors; this family of polypeptides has variously been called the cytokine receptor family or the hematopoietin receptor family. There are other families of receptors that bind cytokines or growth factors, such as the IL-1 receptor family, the TNF receptor family, and the EGF receptor family, but the hematopoietin receptor family is considered to be a distinct group or family of receptors based on certain characteristic structural features or motifs that are shared by members of this family. Some of the members of the hematopoietin receptor family are gp130, the granulocyte colony-stimulating factor receptor (GCSFR), leukemia inhibitory factor receptor (LIF-R), the alpha chains and the common beta chain of the IL-3 and IL-5 receptors, etc.; the hematopoietin receptor family contains more than 20 different polypeptides.

Common structural features of the hematopoietin receptor family of polypeptides include at least one extracellular cytokine receptor domain, which usually contains four cysteines and a WSXWS motif (where W is tryptophan, S is serine, and X indicates any amino acid), and, in most members of the family, a transmembrane and a cytoplasmic domain. The extracellular cytokine receptor domain is involved in ligand-binding activity, and the intracellular domain of a ‘signaling’ subfamily of hematopoietin receptors has a signal transduction function, transmitting the signal generated by ligand binding to a signal transduction pathway that results in the expression of genes involved in cell proliferation, differentiation, and/or activation. These activities of the hematopoietin receptor polypeptide family are mediated through interactions with cytokine ligands and other ligand-binding receptor molecules, with ligand binding to the cytokine receptor domain of hematopoietin receptor polypeptides and facilitating homo- or heterotypic interactions between receptor polypeptides, bringing the cytoplasmic domains of receptors into proximity with each other. Many of the cytokine ligands (such as IL-2, IL-6, or ciliary neurotrophic factor or CNTF, for example) interact with more than one type of heteromeric hematopoietin receptor complex, often with differing affinities, and “common” hematopoietin receptor polypeptides such as gp130 are involved in several different heteromeric receptor complexes that bind a variety of ligands. Because of their ligand-binding and intracellular signaling activities, hematopoietin receptor polypeptides are associated with a wide variety of conditions involving cytokine-influenced cell proliferation, differentiation, or activation. For example, interaction of the gp130 hematopoietin receptor polypeptide with its binding partners is involved in the normal upregulation of cardiac myocyte proliferation (“hypertrophy”) in response to biomechanical stress on the heart, as lack of gp130 leads to heart failure under those conditions (Hirota et al., 1999, Cell 97(2): 189-198). Hematopoietin receptors are also involved in the activation or stimulation of cells in response to environmental factors, for example the activation of hepatocytes in the acute-phase inflammatory response to injury (Taga and Kishimoto, 1992, Crit Rev Immunol. 11(5): 265-280; Neben and Turner, 1993, Stem Cells 11 Suppl 2: 156-162).

Hematopoietin receptor family polypeptides generally are constitutively expressed in many different cell types throughout development, but the expression levels of hematopoietin receptor polypeptides may be up- or downregulated in response to stimuli, and some members of the family exhibit more restricted patterns of expression in particular tissues.

Characteristics and activities of the hematopoietin receptor polypeptide family are described further in the following references, which are incorporated by reference herein: Drachman and Kaushansky, 1995, Curr Opin Hematol. 2(1): 22-28; Ihle, 1995, Nature 377(6550): 591-594; Taga and Kishimoto, 1995, Curr Opin Immunol. 7(1): 17-23; Ihle et al., 1995, Annu Rev Immunol. 13: 369-398; Theze, 1994, Eur Cytokine Netw. 5(4): 353-368; Ihle et al., 1994, Signaling by the cytokine receptor superfamily: JAKs and STATs, Trends Biochem Sci. 19(5): 222-227; Cosman, 1993, Cytokine 5(2): 95-106; and Onishi et al., 1998, Int Rev Immunol. 16(5-6): 617-634.

In order to develop more effective treatments for disorders such as neurological, cardiac, hematopoietic, immunological, hepatic, and pulmonary conditions and diseases involving cell proliferation, differentiation, or activation, including neoplastic transformation or proliferation of virus-infected or cancerous cells, information is needed about previously unidentified members of the hematopoietin receptor polypeptide family.

### SUMMARY OF THE INVENTION

The present invention is based upon the discovery of new human hematopoietin receptor family members, HPR1 and HPR2.

The invention provides an isolated polypeptide consisting of, consisting essentially of, or more preferably, comprising an amino acid sequence selected from the group consisting of:

(a) the amino acid sequence of SEQ ID NO: 4;

(b) amino acids 56 through 77 of SEQ ID NO: 1;

(c) an amino acid sequence selected from the group consisting of: amino acids 1 through 55 of SEQ ID NO: 1; amino acids 5 through 40 of SEQ ID NO: 2; amino acids 1 through 32 of SEQ ID NO: 4; amino acids 1 through 241 of SEQ ID NO: 4; amino acids 1 through 525 of SEQ ID NO: 4; amino acids 20 through 32 of SEQ ID NO: 4; amino acids 33 through 134 of SEQ ID NO: 4; amino acids Xaa1 through Xaa2 of SEQ ID NO: 4, wherein Xaa1 is selected from the group consisting of amino acids 33 through 43 of SEQ ID NO: 4 and Xaa2 is selected from the group consisting of amino acids 228 through 241 of SEQ ID NO: 4; amino acids 33 through 238 of SEQ ID NO: 4; amino acids 33 through 241 of SEQ ID NO: 4; amino acids 33 through 525 of SEQ ID NO: 4; amino acids 33 through 745 of SEQ ID NO: 4; amino acids 44 through 94 of SEQ ID NO: 4; amino acids 139 through 241 of SEQ ID NO: 4; amino acids 242 through 326 of SEQ ID NO: 4; amino acids 242 through 514 of SEQ ID NO: 4; amino acids 337 through 419 of SEQ ID NO: 4; amino acids 433 through 514 of SEQ ID NO: 4; amino acids 526 through 556 of SEQ ID NO: 4; amino acids 533 through 552 of SEQ ID NO: 4; amino acids 553 through 745 of SEQ ID NO: 4; amino acids 557 through 745 of SEQ ID NO: 4; amino acids 563 through 573 of SEQ ID NO: 4; amino acids 563 through 641 of SEQ ID NO: 4; amino acids 567 through 581 of SEQ ID NO: 4; amino acids 588 through 639 of SEQ ID NO: 4; and amino acids 631 through 641 of SEQ ID NO: 4;

(d) fragments of the amino acid sequences of any of (a)-(c) comprising at least 20 contiguous amino acids;

(e) fragments of the amino acid sequences of any of (a)-(c) comprising at least 30 contiguous amino acids;

(f) fragments of the amino acid sequences of any of (a)-(c) having HPR1 polypeptide activity;

(g) fragments of the amino acid sequences of any of (a)-(c) comprising cytokine receptor domain amino acid sequences;

(h) an allelic variant of any of (a)-(c);

(i) amino acid sequences comprising at least 20 amino acids and sharing amino acid identity with the amino acid sequences of any of (a)-(h), wherein the percent amino acid identity is selected from the group consisting of: at least 70%, at least 75%, at least 80%, at least 85%, at least 90%, at least 95%, at least 97.5%, at least 99%, and at least 99.5%;

(j) an amino acid sequence of any of (a)-(i) wherein the polypeptide comprising said amino acid sequence also comprises an amino acid sequence selected from the group consisting of SEQ ID NO: 10, SEQ ID NO: 11, amino acids 652 though 745 of SEQ ID NO: 4, a fragment of the sequence of amino acids 652 though 745 of SEQ ID NO: 4 comprising at least 20 contiguous amino acids; a fragment of the sequence of amino acids 652 though 745 of SEQ ID NO: 4 comprising at least 30 contiguous amino acids; a fragment of the sequence of amino acids 652 though 745 of SEQ ID NO: 4 that is at least 25% of the length of the sequence of amino acids 652 though 745 of SEQ ID NO: 4; a fragment of the sequence of amino acids 652 though 745 of SEQ ID NO: 4 that is at least 50% of the length of the sequence of amino acids 652 though 745 of SEQ ID NO: 4; and a fragment of the sequence of amino acids 652 though 745 of SEQ ID NO: 4 comprising at least one tyrosine residue;

(k) an amino acid sequence of any of (a)-(j) wherein the polypeptide comprising said amino acid sequence does not comprise an amino acid sequence selected from the group consisting of amino acids 239 through 252 of SEQ ID NO: 13; amino acids 643 through 652 of SEQ ID NO: 14; and amino acids 652 through 662 of SEQ ID NO: 15;

(l) an amino acid sequence of (i)-(k), wherein a polypeptide comprising said amino acid sequence of (i)-(k) binds to an antibody that also binds to a polypeptide comprising an amino acid sequence of any of (a)-(h); and

(m) an amino acid sequence of (i)-(l) having HPR1 polypeptide activity.

Preferably, such polypeptides are isolated HPR1 polypeptides or isolated polypeptides having HPR1 polypeptide activity.

Other aspects of the invention are isolated nucleic acids encoding polypeptides of the invention, with a preferred embodiment being an isolated nucleic acid consisting of, consisting essentially of, or more preferably, comprising a nucleotide sequence selected from the group consisting of:

(a) SEQ ID NO: 3;

(b) SEQ ID NO: 5;

(c) nucleotides 132 through 2366 of SEQ ID NO: 3; and

(d) allelic variants of (a)-(c).

An additional preferred embodiment of the invention is an isolated nucleic acid consisting of, consisting essentially of, or more preferably, comprising a nucleotide sequence selected from the group consisting of nucleotides 1 through 137 of SEQ ID NO: 3, nucleotides 138 through 228 of SEQ ID NO: 3, nucleotides 229 through 346 of SEQ ID NO: 3, nucleotides 347 through 528 of SEQ ID NO: 3, nucleotides 529 through 680 of SEQ ID NO: 3, nucleotides 681 through 846 of SEQ ID NO: 3, nucleotides 847 through 926 of SEQ ID NO: 3, nucleotides 927 through 1143 of SEQ ID NO: 3, nucleotides 1144 through 1326 of SEQ ID NO: 3, nucleotides 1327 through 1428 of SEQ ID NO: 3, nucleotides 1429 through 1575 of SEQ ID NO: 3, nucleotides 1576 through 1716 of SEQ ID NO: 3, nucleotides 1717 through 1810 of SEQ ID NO: 3, nucleotides 1811 through 1892 of SEQ ID NO: 3, and nucleotides 1893 through 2480 of SEQ ID NO: 3.

The invention provides an isolated polypeptide consisting of, consisting essentially of, or more preferably, comprising an amino acid sequence selected from the group consisting of:

(a) the amino acid sequence of SEQ ID NO: 21;

(b) an amino acid sequence selected from the group consisting of: amino acids 1 through 177 of SEQ ID NO: 16; amino acids 216 through 245 of SEQ ID NO: 16; SEQ ID NO: 17; SEQ ID NO: 18; and amino acids 349 through 356 of SEQ ID NO: 25;

(c) an amino acid sequence selected from the group consisting of: amino acids 1 through 23 of SEQ ID NO: 21; amino acids 1 through 124 of SEQ ID NO: 21; amino acids 1 through 318 of SEQ ID NO: 21; amino acids 1 through 331 of SEQ ID NO: 21; amino acids 1 through 355 of SEQ ID NO: 21; amino acids Xaa1 through Xaa2 of SEQ ID NO: 21, wherein Xaa1 is selected from the group consisting of amino acids 24 through 30 of SEQ ID NO: 21 and Xaa2 is selected from the group consisting of amino acids 115 through 124 of SEQ ID NO: 21; amino acids 24 through 124 of SEQ ID NO: 21; amino acids 24 through 331 of SEQ ID NO: 21; amino acids 24 through 355 of SEQ ID NO: 21; amino acids Xaa3 through Xaa4 of SEQ ID NO: 21, wherein Xaa3 is selected from the group consisting of amino acids 125 through 133 of SEQ ID NO: 21 and Xaa4 is selected from the group consisting of amino acids 309 through 331 of SEQ ID NO: 21; amino acids 125 through 219 of SEQ ID NO: 21; amino acids 125 through 331 of SEQ ID NO: 21; amino acids 133 through 309 of SEQ ID NO: 21; amino acids 224 through 320 of SEQ ID NO: 21; amino acids 224 through 331 of SEQ ID NO: 21; amino acids 319 through 565 of SEQ ID NO: 21; amino acids Xaa5 through Xaa6 of SEQ ID NO: 21, wherein Xaa5 is selected from the group consisting of amino acids 376 through 393 of SEQ ID NO: 21 and Xaa6 is selected from the group consisting of amino acids 618 through 629 of SEQ ID NO: 21; amino acids 376 through 629 of SEQ ID NO: 21; amino acids 393 through 440 of SEQ ID NO: 21; amino acids 393 through 618 of SEQ ID NO: 21; and amino acids 397 through 611 of SEQ ID NO: 21;

(d) fragments of the amino acid sequences of any of (a)-(c) comprising at least 20 contiguous amino acids;

(e) fragments of the amino acid sequences of any of (a)-(c) comprising at least 30 contiguous amino acids;

(f) fragments of the amino acid sequences of any of (a)-(c) having HPR2 polypeptide activity;

(g) fragments of the amino acid sequences of any of (a)-(c) comprising cytokine receptor domain amino acid sequences;

(h) an allelic variant of any of (a)-(c);

(i) amino acid sequences comprising at least 20 amino acids and sharing amino acid identity with the amino acid sequences of any of (a)-(h), wherein the percent amino acid identity is selected from the group consisting of: at least 70%, at least 75%, at least 80%, at least 85%, at least 90%, at least 95%, at least 97.5%, at least 99%, and at least 99.5%;

(j) an amino acid sequence of any of (a)-(i) wherein the polypeptide comprising said amino acid sequence also comprises an amino acid sequence selected from the group consisting of: amino acids 1 through 177 of SEQ ID NO: 16; amino acids 216 through 245 of SEQ ID NO: 16; SEQ ID NO: 17; SEQ ID NO: 18; amino acids 349 through 356 of SEQ ID NO: 25; amino acids 319 through 565 of SEQ ID NO: 21; amino acids Xaa5 through Xaa6 of SEQ ID NO: 21, wherein Xaa5 is selected from the group consisting of amino acids 376 through 393 of SEQ ID NO: 21 and Xaa6 is selected from the group consisting of amino acids 618 through 629 of SEQ ID NO: 21; amino acids 376 through 629 of SEQ ID NO: 21; amino acids 393 through 440 of SEQ ID NO: 21; amino acids 393 through 618 of SEQ ID NO: 21; amino acids 397 through 611 of SEQ ID NO: 21; amino acids 381 though 629 of SEQ ID NO: 21; a fragment of the sequence of amino acids 381 though 629 of SEQ ID NO: 21 comprising at least 20 contiguous amino acids; a fragment of the sequence of amino acids 381 though 629 of SEQ ID NO: 21 comprising at least 30 contiguous amino acids; a fragment of the sequence of amino acids 381 though 629 of SEQ ID NO: 21 that is at least 25% of the length of the sequence of amino acids 381 though 629 of SEQ ID NO: 21; a fragment of the sequence of amino acids 381 though 629 of SEQ ID NO: 21 that is at least 50% of the length of the sequence of amino acids 381 though 629 of SEQ ID NO: 21; a fragment of the sequence of amino acids 381 though 629 of SEQ ID NO: 21 comprising at least one of the following: an HPR2 Box 1 motif, an HPR2 Box 2 motif, and an HPR2 Box 3 motif; and a fragment of the sequence of amino acids 381 though 629 of SEQ ID NO: 21 comprising at least one tyrosine residue;

(k) an amino acid sequence of any of (a)-() wherein the polypeptide comprising said amino acid sequence does not comprise amino acids 381 through 384 of SEQ ID NO: 26;

(l) an amino acid sequence of (i)-(k), wherein a polypeptide comprising said amino acid sequence of (i)-(k) binds to an antibody that also binds to a polypeptide comprising an amino acid sequence of any of (a)-(h); and

(m) an amino acid sequence of (i)-(l) having HPR2 polypeptide activity.

Preferably, such polypeptides are isolated HPR2 polypeptides or isolated polypeptides having HPR2 polypeptide activity.

Other aspects of the invention are isolated nucleic acids encoding polypeptides of the invention, with a preferred embodiment being an isolated nucleic acid consisting of, consisting essentially of, or more preferably, comprising a nucleotide sequence selected from the group consisting of:

(a) SEQ ID NO: 19;

(b) SEQ ID NO: 20;

(c) SEQ ID NO: 22;

(d) SEQ ID NO: 24; and

(d) allelic variants of (a)-(d).

An additional preferred embodiment of the invention is an isolated nucleic acid consisting of, consisting essentially of, or more preferably, comprising a nucleotide sequence selected from the group consisting of nucleotides 107 through 175 of SEQ ID NO: 19, nucleotides 107 through 478 of SEQ ID NO: 19, nucleotides 107 through 1060 of SEQ ID NO: 19, nucleotides 107 through 1099 of SEQ ID NO: 19, nucleotides 107 through 1171 of SEQ ID NO: 19, nucleotides 176 through 478 of SEQ ID NO: 19, nucleotides 176 through 1099 of SEQ ID NO: 19, nucleotides 176 through 1171 of SEQ ID NO: 19, nucleotides 479 through 763 of SEQ ID NO: 19, nucleotides 479 through 1099 of SEQ ID NO: 19, nucleotides 503 through 1033 of SEQ ID NO: 19, nucleotides 776 through 1066 of SEQ ID NO: 19, nucleotides 776 through 1099 of SEQ ID NO: 19, nucleotides 1061 through 1801 of SEQ ID NO: 19, nucleotides 1232 through 1993 of SEQ ID NO: 19, nucleotides 1283 through 1426 of SEQ ID NO: 19, nucleotides 1283 through 1960 of SEQ ID NO: 19, and nucleotides 1295 through 1939 of SEQ ID NO: 19.

The invention also provides isolated genomic nucleic acids corresponding to the nucleic acids of the invention.

Another aspect of the invention provides isolated nucleic acids, preferably having a length of at least 15 nucleotides, that hybridize under conditions of moderate stringency to the nucleic acids encoding polypeptides of the invention. In preferred embodiments of the invention, such nucleic acids encode a polypeptide having HPR1 and/or HPR2 polypeptide activity, or comprise a nucleotide sequence that shares nucleotide sequence identity with the nucleotide sequences of the nucleic acids of the invention, wherein the percent nucleotide sequence identity is selected from the group consisting of: at least 70%, at least 75%, at least 80%, at least 85%, at least 90%, at least 95%, at least 97.5least 99%, and at least 99.5%.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. A preferred process provided by the invention further comprises purifying said polypeptide. In another aspect of the invention, the polypeptide produced by said process is provided.

Further aspects of the invention are isolated antibodies that bind to the polypeptides of the invention, preferably monoclonal antibodies, also preferably humanized antibodies or humanized antibodies, and preferably wherein the antibody inhibits the activity of said polypeptides.

The invention additionally provides a method of designing an inhibitor of the polypeptides of the invention, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

In a further aspect of the invention, a method is provided for identifying compounds that alter HPR1 and/or HPR2 polypeptide activity comprising

(a) mixing a test compound with a polypeptide of the invention; and

(b) determining whether the test compound alters the HPR1 and/or HPR2 polypeptide activity of said polypeptide.

In another aspect of the invention, a method is provided identifying compounds that inhibit the binding activity of HPR1 and/or HPR2 polypeptides comprising

(a) mixing a test compound with a polypeptide of the invention and a binding partner of said polypeptide; and

(b) determining whether the test compound inhibits the binding activity of said polypeptide.

In preferred embodiments, the binding partner is a four alpha helix bundle cytokine; more preferably, the binding partner is selected from the group consisting of IL-6, OSM, LIF, CNTF, CLC, IL-12p35, and IL-23p19, and most preferably the binding partners are a soluble hematopoietin receptor such as EBI-3, soluble IL-6R alpha, cytokine-like factor-1 (CLF), IL-12p40, or a soluble form of HPR1 and/or HPR2 in conjunction with a four alpha helix bundle cytokine.

The invention also provides a method for increasing ligand-binding activity, comprising providing at least one compound selected from the group consisting of the polypeptides of the invention and agonists of said polypeptides; with a preferred embodiment of the method further comprising increasing said activity in a patient by administering at least one polypeptide of the invention.

Further provided by the invention is a method for decreasing ligand-binding activity, comprising providing at least one antagonist of the polypeptides of the invention; with a preferred embodiment of the method further comprising decreasing said activity in a patient by administering at least one antagonist of the polypeptides of the invention, and with a further preferred embodiment wherein the antagonist is an antibody that inhibits the activity of any of said polypeptides.

The invention additionally provides a method for treating a cell proliferation condition comprising administering at least one compound selected from the group consisting of the polypeptides of the invention and agonists of said polypeptides; with a preferred embodiment wherein the cell proliferation condition is selected from the group consisting of pancytopenia, leukopenia, anemia, thrombocytopenia, neurodegenerative disorders, and osteoporosis resulting from a lack of bone-forming cells.

The invention additionally provides a method for treating a metabolic condition comprising administering at least one compound selected from the group consisting of the polypeptides of the invention and agonists of said polypeptides; with a preferred embodiment wherein the metabolic condition is obesity.

The invention additionally provides a method for treating a reproductive hormone condition comprising administering at least one compound selected from the group consisting of the polypeptides of the invention and agonists of said polypeptides; with a preferred embodiment wherein the condition is selected from the group consisting of deficient mammary development and infertility.

In other aspects of the invention, a method is provided for treating a cell proliferation condition comprising administering an antagonist of the polypeptide of the invention; with a preferred embodiment wherein the cell proliferation condition is selected from the group consisting of leukemia, tumour metastasis, and osteoporosis resulting from an excess of bone-resorbing cells.

In other aspects of the invention, a method is provided for treating a metabolic condition comprising administering an antagonist of the polypeptide of the invention; with a preferred embodiment wherein the metabolic condition is selected from the group consisting of cachexia, wasting, and AIDS-related weight loss.

In other aspects of the invention, a method is provided for treating cancer conditions stimulated by reproductive hormones comprising administering an antagonist of the polypeptide of the invention; with a preferred embodiment wherein the condition is selected from the group consisting of breast cancer and prolactinoma.

In another embodiment of the invention, methods are provided for using HPR1 and HPR2 polypeptides and antagonists thereof as adjuvants.

A further embodiment of the invention provides a use for the polypeptides of the invention in the preparation of a medicament for treating a cell proliferation condition; with a preferred embodiment wherein the cell proliferation condition is selected from the group consisting of pancytopenia, leukopenia, anemia, thrombocytopenia, neurodegenerative disorders, and osteoporosis.

A further embodiment of the invention provides a use for the polypeptides of the invention in the preparation of a medicament for treating a metabolic condition; with a preferred embodiment wherein the metabolic condition is obesity.

A further embodiment of the invention provides a use for the polypeptides of the invention in the preparation of a medicament for treating a reproductive hormone condition; with a preferred embodiment wherein the condition is selected from the group consisting of deficient mammary development and infertility.

### DETAILED DESCRIPTION OF THE INVENTION

Similarities of HPR1 and HPR2 Structure to Other Hematopoietin Receptor Family Members

We have identified HPR1 and HPR2, new human hematopoietin receptor polypeptides having structural features characteristic of this polypeptide family; the amino acid sequence of an HPR1 polypeptide is provided in SEQ ID NO: 4 and the amino acid sequence of three alternatively spliced forms of HPR2 polypeptide are provided in SEQ ID NOs 21, 23, and 25. We have also identified the murine homologue of human HPR1; the amino acid sequence of Mus musculus HPR1 is presented in SEQ ID NO: 12. (The use of “HPR1” without a species designation refers to HPR1 polypeptides generally, for example, human and/or murine, mammalian, or vertebrate HPR1 polypeptides.) Alignments showing the sequence similarities between HPR1, HPR2, and other hematopoietin receptors are presented in Tables 1, 2, and 3 in Example 1 below.

The typical structural elements common to members of the hematopoietin receptor polypeptide family include an extracellular region comprising at least one cytokine receptor domain, and in most members of the family, a cytoplasmic region that in at least a subset of the hematopoietin receptor polypeptides comprises domains involved in intracellular signaling functions. A signal sequence is found at the N-terminus of hematopoietin receptor family polypeptides, and is followed, in N-to-C order, by an immunoglobulin (Ig)-like domain (in some members of the family), a cytokine receptor domain, three copies of a fibronectin repeat (in some members of the family), a transmembrane domain or a glycosyl-phosphatidyl inositol (GPI) linkage to the membrane (except in soluble members of the family, which in most cases are soluble splice variant forms of transmembrane or membrane-linked hematopoietin receptor polypeptides), and a cytoplasmic domain (which is not present in soluble forms). The extracellular domain of hematopoietin receptor polypeptides extends from the N terminus to the transmembrane domain of the protein, and includes the cytokine receptor domain and any Ig-like domains (approximately 100 amino acids in length) or fibronectin repeats (such as fibronectin type III repeats which are approximately 81-83 amino acids in length and are separated by spacer sequences of approximately 10 to 13 amino acids) that may be present in certain of the hematopoietin receptor polypeptides. There are key residues within the cytokine receptor domain, the two or four conserved cysteine residues and the WSXWS motif; substitutions of these residues are likely to be associated with an altered function or lack of that function for the polypeptide. The cytokine receptor domain, which is approximately 200 amino acids in length, can be subdivided into two roughly equal subdomains—an N-terminal ‘conserved cysteine’ domain and a more C-terminal ‘WSXWS’ domain—separated by a proline-rich ‘linker’ stretch of four amino acids that allows the two subdomains to form a ligand binding site between them (Bravo and Heath, 2000, EMBO J. 19(11): 2399-2411).

The intracellular domain (also called “cytoplasmic domain”) of the hematopoietin receptor polypeptides (in those family members that contain such a domain), extends from the transmembrane domain of the protein to the C terminus, and in the signaling receptor subgroup, includes regions involved in intracellular signal transduction functions. Although the amino acid sequence of the intracellular domain varies considerably between hematopoietin receptor polypeptides, there are a few regions that show some similarity between the members of the family and which have been determined to be involved in binding to members of the signal transduction cascade. “Box 1” is a stretch of 9 to 12 amino acids that begins about 9 amino acids C-terminal to the transmembrane domain, and has within it a conserved Ar-P-X-Al-P-X-P motif, where Ar is an aromatic amino acid (Trp, Phe, or Tyr) and Al is an aliphatic amino acid (Ala, Gly, Val, Leu, or Ile). About 8 amino acids C-terminal to Box 1 there is a conserved aromatic amino acid (usually Trp but also Phe or Tyr), and approximately 15 to 60 amino acids further C-terminal there is a motif of about 11 to 13 amino acids, “Box 2”. While Box 1 is present in most of the hematopoietin receptor polypeptides, the Box 2 motif is present in a subset of the hematopoietin receptor family including gp130, GCSFR, LIF-R, the erythropoietin receptor (EPO-R), and several others. Mutations to residues within Box 1 or Box 2, or to the conserved aromatic residue between the Box 1 and Box 2 motifs, have inactivated the ability of the mutated receptor to stimulate cell proliferation upon the addition of ligand. A further conserved domain has been identified in the cytoplasmic domains of signaling cytokine receptors such as gp130, LIF-R, and G-CSFR: “Box 3”. The Box 3 motif is about 10 to 15 amino acids located between approximately 70 and 150 amino acids C-terminal of the transmembrane domain, and has a rough match to a (P/T)VXGXGYXXQ consensus sequence. Cytoplasmic regions of these receptors containing Box 3 have been associated with a macrophage differentiation promoting activity (in the case of gp130) and a granulocyte differentiation promoting activity (in the case of G-CSFR) (Soede-Bobok and Touw, 1997, J Mol Med 75: 470-477); however, members of the LIF/IL-6 gp130-sharing family of hematopoietin receptors can also be involved in suppression of differentiation (see Ernst et al., 1999, J Biol Chem 274(14): 9729-9737). Finally, the cytoplasmic domains of signaling hematopoietin receptor polypeptides contain several tyrosine residues that are potential sites for phosphorylation. Although hematopoietin receptors themselves do not generally have a protein kinase activity, they interact with and are phosphorylated by kinases within the JAK/STAT signal transduction pathways. Mutations in the Box 1 motif abolish the ability of certain of the signaling hematopoietin receptors to bind members of the Janus kinase (JAK) family, particularly JAK2 or JAK1 (Taner et al., 1995, J Biol Chem 270(12): 6523-6530). Hematopoietin receptor-ligand interactions also activate the ERK/MAPK pathway, most likely through the phosphorylation of tyrosine residues in the cytoplasmic domains as the tyrosines at cytoplasmic positions 118 of gp130 (amino acid 759 of SEQ ID NO: 8) and 115 of LIF-R (amino acid 974 of SEQ ID NO: 6) are present within SHP2 binding sites (Schiemann et al., 1997, J Biol Chem 272(26): 16631-16636). The cytoplasmic tyrosine residues of signaling hematopoietin receptors and the amino acids around them are also important motifs for the recruitment and phosphorylation of signal-transducing STAT polypeptides (Hirano et al., 2000, Oncogene 19: 2548-2556).

Human HPR1 polypeptide has a signal sequence extending from approximately amino acid 20 through amino acid 32 of SEQ ID NO: 4, with the mature polypeptide produced by cleavage of this signal sequence predicted to have an amino acid sequence beginning at amino acid 33 of SEQ ID NO: 4. Human HPR1 has a cytokine receptor domain extending approximately from amino acid 33 through amino acid 241 of SEQ ID NO: 4; three fibronectin repeats from approximately amino acid 242 of SEQ ID NO: 4 to about amino acid 515 of SEQ ID NO: 4; a transmembrane domain that begins approximately between amino acids 526 and 533 of SEQ ID NO: 4 and extends to approximately between amino acids 552 and 556 of SEQ ID NO: 4 (defining a smaller ‘core’ transmembrane domain from amino acid 533 to amino acid 552 of SEQ ID NO: 4 and an extended transmembrane domain from amino acid 526 to amino acid 556 of SEQ ID NO: 4); and a cytoplasmic domain extending from the end of the transmembrane domain (i.e. beginning roughly between amino acids 553 and 557 of SEQ ID NO: 4) and extending through the carboxyl terminus of the polypeptide (amino acid 745 of SEQ ID NO: 4). Therefore, human HPR1 polypeptide has an overall structure consistent with other hematopoietin receptor family members. The four conserved cysteine residues within the human HPR1 cytokine receptor domain are located at positions 43, 53, 81, and 94 of SEQ ID NO: 4, and the human HPR1 WSXWS motif is located from amino acid 224 through amino acid 228 of SEQ ID NO: 4. The human HPR1 N-terminal cytokine receptor subdomain containing four conserved cysteine residues extends approximately from amino acid 33 of SEQ ID NO: 4 to amino acid 134 of SEQ ID NO: 4; the proline-rich linker is amino acids 135 through 138 of SEQ ID NO: 4; and the WSXWS-containing C-terminal cytokine receptor subdomain extends from amino acid 139 to about amino acid 241 of SEQ ID NO: 4. In human HPR1, as in several members of the hematopoietin receptor family, the cytokine receptor domain is followed by three fibronectin type III repeats; these repeats are located within the human HPR1 amino acid sequence of SEQ ID NO: 4 at the following approximate locations: amino acids 242 to 244 through 324 to 326, amino acids 336 to 337 through 419 to 422, and amino acids 430 to 433 through 514 to 515. Within its intracellular domain, human HPR1 polypeptide contains a good match to the Box 1 conserved motif from amino acid 563 through amino acid 573 of SEQ ID NO: 4, a conserved downstream Trp residue (amino acid 581 of SEQ ID NO: 4), and a Box 2 motif from amino acid 631 to amino acid 641 of SEQ ID NO: 4. The cytoplasmic domains of signaling hematopoietin receptor polypeptides contain several tyrosine residues that are potential sites for phosphorylation; in human HPR1, such tyrosines are located at positions 652, 683, and 721 of SEQ ID NO: 4. Human HPR1 contains several instances of an Asp-containing motif within its cytoplasmic region. In the area overlapping the Box 2 location, human HPR1 has repeated amino acid sequences as shown in the following table; these sequences form a consensus sequence of DKL(N/V)(T/Al), where Al is an aliphatic residue as described above. Other signaling hematopoietin receptors such as murine HPR1 (at amino acids 600 through 604 of SEQ ID NO: 12) and gp130 also contain at least one similar Asp-containing sequence in the region around and following the Box 2 location.

| Repeat Sequence | Location in SEQ ID NO:4 |
|---|---|
| DKLNL | amino acids 588 through 592 |
| DSVNT | amino acids 597 through 601 |
| DRILK | amino acids 603 through 607 |
| DKLVI | amino acids 614 through 618 |
| DKLVV | amino acids 619 through 623 |
| DEART | amino acids 635 through 639 |

Variants, presumably splice variants, of human HPR1 are described in WO 00/75314: a 252-amino-acid form (“NR10.2”), a 652-amino-acid form (“NR10.1”), and a 662-amino-acid form (“NR1O.3”). The 252-amino-acid form of HPR1 (SEQ ID NO: 13) is identical to SEQ ID NO: 4 through amino acid 238, and then has a divergent amino acid sequence from amino acid 239 through 252 of SEQ ID NO: 13. This 252-amino-acid form of human HPR1 therefore does not contain the fibronectin type III repeats found in the full-length 745-amino-acid HPR1 of SEQ ID NO: 4, or the transmembrane domain or the intracellular region of the SEQ ID NO: 4 polypeptide. The 652-amino-acid form of HPR1 (SEQ ID NO: 14) is identical to SEQ ID NO: 4 through amino acid 642, and then has a divergent amino acid sequence from amino acid 643 through 652 of SEQ ID NO: 14.; and the 662-amino-acid form of HPR1 (SEQ ID NO: 15) is identical to SEQ ID NO: 4 through amino acid 651, and then has a divergent amino acid sequence from amino acid 652 through 662 of SEQ ID NO: 15. The 652- and 662-amino-acid forms of human HPR1 therefore do not contain the tyrosine residues at positions 652, 683, and 721 of the intracellular region of the SEQ ID NO: 4 polypeptide which are potential substrates for phosphorylation by kinases, such as those of the ERK/MAPK signaling pathways.

The Mus musculus HPR1 amino acid sequence of SEQ ID NO: 12 has a signal sequence beginning approximately between amino acid 13 and amino acid 16 of SEQ ID NO: 12 and extending approximately through amino acid 28 of SEQ ID NO: 12, with the mature polypeptide produced by cleavage of this signal sequence predicted to have an amino acid sequence beginning at amino acid 29 of SEQ ID NO: 12. Murine HPR1 has a cytokine receptor domain extending approximately from amino acid 29 through amino acid 224 of SEQ ID NO: 12; three fibronectin repeats from approximately amino acid 225 of SEQ ID NO: 12 to about amino acid 499 of SEQ ID NO: 12; a transmembrane domain that begins approximately between amino acids 510 and 517 of SEQ ID NO: 12 and extends to approximately between amino acids 532 and 533 of SEQ ID NO: 12 (defining a smaller ‘core’ transmembrane domain from amino acid 517 to amino acid 532 of SEQ ID NO: 12 and an extended transmembrane domain from amino acid 510 to amino acid 533 of SEQ ID NO: 12); and a cytoplasmic domain extending from the end of the transmembrane domain (i.e. beginning roughly between amino acids 533 and 534 of SEQ ID NO: 12) and extending through the carboxyl terminus of the polypeptide (amino acid 726 of SEQ ID NO: 12). Therefore, murine HPR1 polypeptide has an overall structure consistent with other hematopoietin receptor family members. There are two conserved cysteine residues within the murine HPR1 cytokine receptor domain located at positions 39 and 49of SEQ ID NO: 12, and there are two additional cysteines in this region (although at non-conserved positions) at amino acids 90 and 97 of SEQ ID NO: 12. The murine HPR1 WSXWS motif is located from amino acid 207 through amino acid 211 of SEQ ID NO: 12. The murine HPR1 N-terminal cytokine receptor subdomain containing two conserved cysteine residues (and two additional cysteine residues) extends approximately from amino acid 29 of SEQ ID NO: 12 to amino acid 124 of SEQ ID NO: 12; the proline-rich linker is amino acids 125 through 128 of SEQ ID NO: 12; and the WSXWS-containing C-terminal cytokine receptor subdomain extends from amino acid 129 to about amino acid 224 of SEQ ID NO: 12. In murine HPR1, as in several members of the hematopoietin receptor family, the cytokine receptor domain is followed by three fibronectin type III repeats; these repeats are located within the murine HPR1 amino acid sequence of SEQ ID NO: 12 at the following approximate locations: amino acids 225 to 227 through 307 to 309 , amino acids 319 to 320 through 403 to 406, and amino acids 417 through 498 to 499. Within its intracellular domain, murine HPR1 polypeptide contains a good match to the Box 1 conserved motif from amino acid 547 through amino acid 557 of SEQ ID NO: 12, a conserved downstream Trp residue (amino acid 565 of SEQ ID NO: 12), and a Box 2 motif from amino acid 612 through amino acid 622 of SEQ ID NO: 12. The cytoplasmic domains of signaling hematopoietin receptor polypeptides contain several tyrosine residues that are potential sites for phosphorylation; in murine HPR1, such tyrosines are located at positions 633, 674, and 701 of SEQ ID NO: 12.

Human HPR2 polypeptide has a signal sequence extending from approximately amino acid 11 through amino acid 23 of SEQ ID NO: 21, with the mature polypeptide produced by cleavage of this signal sequence predicted to have an amino acid sequence beginning at amino acid 24 of SEQ ID NO: 21. The membrane-spanning (629 amino acids) form of HPR2 has an N-terminal Ig-like domain extending approximately from amino acid 24 through amino acid 124 of SEQ ID NO: 21, a cytokine receptor domain extending approximately from amino acid 125 through an amino acid from 320 to 331 of SEQ ID NO: 21; a transmembrane domain that begins approximately at amino acid 356 of SEQ ID NO: 21 and extends to approximately amino acid 375 of SEQ ID NO: 21; and a cytoplasmic domain extending from the end of the transmembrane domain (i.e. beginning approximately at amino acid 376 of SEQ ID NO: 21) and extending through the carboxyl terminus of the polypeptide (amino acid 629 of SEQ ID NO: 21). Therefore, HPR2 polypeptide has an overall structure consistent with other hematopoietin receptor family members. The N-terminal Ig-like domain contains six cysteine residues at positions 30, 52, 59, 101, 105, and 115 of SEQ ID NO: 21, the most conserved of which appear to be the two cysteines at positions 52 and 101; the cysteines at positions 30, 115 (and to a lesser extent, at 105) also align with cysteines at similar positions in Ig or Ig-like domains. The HPR2 Ig-like domain appears to have the greatest degree of sequence similarity with members of the LIR (leukocyte Ig-like receptor) polypeptide family, particularly LIR-3 and LIR-4. The two conserved cysteine residues within the human HPR2 cytokine receptor domain are located at amino acid positions 133 and 144 of SEQ ID NO: 21, and the HPR2 version of the WSXWS motif, which has a glutamine residue at the second position of the motif rather than a serine residue, is located from amino acid 304 through amino acid 308 of SEQ ID NO: 21. The HPR2 N-terminal cytokine receptor subdomain containing the two conserved cysteine residues extends approximately from amino acid 125 of SEQ ID NO: 21 to amino acid 219 of SEQ ID NO: 21; the proline-rich linker (in this case, proline- and alanine-rich) is amino acids 220 through 223 of SEQ ID NO: 21; and the ‘WQXWS’-containing C-terminal cytokine receptor subdomain extends from amino acid 224 through an amino acid from 320 to 331 of SEQ ID NO: 21. HPR2 does not contain the fibronectin type III repeats found in human and murine HPR1. Within its intracellular domain, the membrane-spanning (629 amino acids) form of HPR2 contains a good match to the Box 1 conserved motif from amino acid 393 through amino acid 403 of SEQ ID NO: 21, does not contain a Trp residue between Box 1 and Box2, and has a Box 2 motif from amino acid 430 to amino acid 440 of SEQ ID NO: 21. There are also two matches to the Box 3 motif in this membrane-spanning HPR2 polypeptide, at amino acids 478 through 491 and at amino acids 605 through 618 of SEQ ID NO: 21. The cytoplasmic domains of signaling hematopoietin receptor polypeptides contain several tyrosine residues that are potential sites for phosphorylation; in human HPR2, such tyrosines are located at amino acid positions 397 (within the Box 1 motif), 429 (immediately N-terminal to the Box 2 motif), 450, 463, and 476 Oust N-terminal of the most N-terminal Box 3 motif), and amino acids 484 and 611 (each of these last two amino acids is within a Box 3 motif) of SEQ ID NO: 21. In several respects, the membrane-spanning form of HPR2 shows similarity to the LIF-R hematopoietin receptor: both of these molecules have an Ig-like domain that is followed by a cytokine receptor domain having two (as compared to four) conserved cysteines; and both have Box 1, Box 2, and Box 3 motifs in their intracellular domains, and do not have a tryptophan residue between Box 1 and Box 2.

The HPR2-ex9 polypeptide of SEQ ID NO: 25 (356 amino acids), created by alternative splicing which removes exon 9 of the HPR2 coding sequence (see Example 1 below), is identical to the HPR2 629-amino-acid form from amino acid 1 through amino acid 348, but then diverges in sequence for the eight amino acids from amino acid 349 to the C terminus at amino acid 356. The HPR2-ex9 form does not contain a transmembrane region, and is expected to be a secreted form of HPR2 containing the HPR2 extracellular Ig-like and cytokine receptor domains. The HPR2-ex8-ex9 polypeptide of SEQ ID NO: 23 (565 amino acids), created by alternative splicing which removes exons 8 and 9 of the HPR2 coding sequence (see Example 1 below), is identical to the HPR2 629-amino-acid form from amino acid 1 through amino acid 318, is missing the next 64 amino acids which include the transmembrane domain, but then shows identity between amino acid 319 through amino acid 565 of SEQ ID NO: 23 and the C-terminal region of the 629-amino-acid form of HPR2. The HPR2-ex8-ex9 form is also expected to be a secreted form of HPR2 containing not only the HPR2 extracellular Ig-like and cytokine receptor domains, but also the C-terminal portion of the HPR2 protein which includes the Box 1, Box 2, and Box 3 motifs. A variant, presumably a splice variant, of human HPR2 is described in WO 00/73451: a 384-amino-acid form (“DCRS2”). This 384-amino-acid form of HPR2 (SEQ ID NO: 26) is identical to SEQ ID NO: 21 through amino acid 380, and then has a divergent amino acid sequence from amino acid 381 through 384 of SEQ ID NO: 26. This 384-amino-acid form of human HPR2 therefore does not contain the intracellular region of the SEQ ID NO: 21 HPR2 polypeptide, which contains the Box1, 2, and 3 motifs and intracellular tyrosine residues that are involved in the signaling (or signal transduction) function of the SEQ ID NO: 21 HPR2 polypeptide.

The Mus musculus HPR2 amino acid sequence of SEQ ID NO: 27 has a signal sequence beginning approximately between amino acid 8 and amino acid 11 and extending through amino acid 23 of SEQ ID NO: 27, with the mature polypeptide produced by cleavage of this signal sequence predicted to have an amino acid sequence beginning at amino acid 24 of SEQ ID NO: 27. Mus musculus HPR2, like the membrane-spanning form of human HPR2, has an N-terminal Ig-like domain extending approximately from amino acid 24 through amino acid 124 of SEQ ID NO: 27, a cytokine receptor domain extending approximately from amino acid 125 through an amino acid from 341 to 350 of SEQ ID NO: 27; a transmembrane domain that begins approximately between amino acid 373 and amino acid 380 of SEQ ID NO: 27 and extends through approximately between amino acid 394 and amino acid 395 of SEQ ID NO: 27 (defining a smaller ‘core’ transmembrane domain from amino acid 380 to amino acid 394 of SEQ ID NO: 27 and an extended transmembrane domain from amino acid 373 to amino acid 395 of SEQ ID NO: 27); and a cytoplasmic domain extending from the end of the transmembrane domain (i.e. beginning approximately at amino acid 395 or at amino acid 396 of SEQ ID NO: 27) and extending through the carboxyl terminus of the polypeptide (amino acid 644 of SEQ ID NO: 27). Therefore, murine HPR2 polypeptide has an overall structure consistent with other hematopoietin receptor family members. The N-terminal Ig-like domain contains six cysteine residues at positions 30, 52, 59, 101, 105, and 115 of SEQ ID NO: 27, the most conserved of which appear to be the two cysteines at positions 52 and 101; the cysteines at positions 30, 115 (and to a lesser extent, at 105) also align with cysteines at similar positions in Ig or Ig-like domains. As with human HPR2, the murine HPR2 Ig-like domain appears to have the greatest degree of sequence similarity with members of the LIR (leukocyte Ig-like receptor) polypeptide family. The two conserved cysteine residues within the human HPR2 cytokine receptor domain are located at amino acid positions 133 and 144 of SEQ ID NO: 27, and the murine HPR2 version of the “WSXWS” motif, which like human HPR2 has a glutamine residue at the second position of the motif rather than a serine residue, is located from amino acid 324 through amino acid 328 of SEQ ID NO: 27. The murine HPR2 polypeptide contains an insert of 20 amino acids relative to the human HPR2 polypeptide; this insert region extends from amino acid 297 through amino acid 316 of SEQ ID NO: 27, and is a perfect repeat of amino acids 317 through 336 of SEQ ID NO: 27. Therefore, in the SEQ ID NO: 27 form of murine HPR2, there is a second WQXWS motif at amino acids 304 through 308 of SEQ ID NO: 27. The murine HPR2 N-terminal cytokine receptor subdomain containing the two conserved cysteine residues extends approximately from amino acid 125 of SEQ ID NO: 27 to amino acid 219 of SEQ ID NO: 27; the proline-rich linker (in this case, proline- and alanine-rich) is amino acids 220 through 223 of SEQ ID NO: 27; and the C-terminal cytokine receptor subdomain containing the two repeats of the WQXWS motif extends from amino acid 224 through an amino acid from 340 to 350 of SEQ ID NO: 27. Murine HPR2 does not contain the fibronectin type III repeats found in human and murine HPR1. Within its intracellular domain, this membrane-spanning form of murine HPR2 contains a good match to the Box 1 conserved motif from amino acid 412 through amino acid 422 of SEQ ID NO: 27, does not contain a Trp residue between Box 1 and Box2, and has a Box 2 motif from amino acid 449 to amino acid 459 of SEQ ID NO: 27. There are also two matches to the Box 3 motif in this murine membrane-spanning HPR2 polypeptide, at amino acids 498 through 511 and at amino acids 620 through 633 of SEQ ID NO: 27. The cytoplasmic domains of signaling hematopoietin receptor polypeptides contain several tyrosine residues that are potential sites for phosphorylation; in murine HPR2, such tyrosines are located at amino acid positions 416 (within the Box 1 motif), 448 (immediately N-terminal to the Box 2 motif), 469, and 496 Oust N-terminal of the most N-terminal Box 3 motif), and amino acids 504 and 626 (each of these last two amino acids is within a Box 3 motif) of SEQ ID NO: 27. There is an additional intracellular tyrosine located at position 542 of SEQ ID NO: 27. As with the membrane-spanning form of human HPR2, murine HPR2 shows similarity to the LIF-R hematopoietin receptor.

Each of the HPR1 and the HPR2 groups of related polypeptides therefore contains a distinct subset of the several features characteristic of at least some members of the hematopoietin receptor family. The skilled artisan will recognize that the boundaries of the regions of the HPR1 and HPR2 polypeptides described above are approximate and that the precise boundaries of such domains, as for example the boundaries of the transmembrane region (which can be predicted by using computer programs available for that purpose), can also differ from member to member within the hematopoietin receptor polypeptide family.

The hematopoietin receptor polypeptide family is highly to moderately conserved between species, with the family members within a particular species exhibiting some sequence conservation, particularly with respect to the conserved domains and residues described above. Subfamilies of the hematopoietin receptor polypeptide family can be defined on the basis of structure, for example the Ig-like domain containing members, or the fibronectin repeat containing members. It is also possible to group hematopoietin receptor polypeptides according to the length of the cytoplasmic domain, with those receptors having a longer cytoplasmic domain being more likely to be signaling receptors. Subgroups of the hematopoietin receptor family can also be defined on the basis of a shared common signaling receptor present in several different combinations of heteromeric receptors. For example, the gp130 signaling receptor is found in separate complexes with LIF-R, IL-6R alpha or a soluble form of IL-6R alpha, and CNTFR alpha; monomeric forms or multimeric combinations of these receptor components bind to IL-6, OSM, LIF, and/or CNTF; thus a “gp130-sharing group” subfamily would include these hematopoietin receptor polypeptides and be associated with this group of cytokines. Another group of hematopoietin receptors are those which associate with a ligand comprising at least two soluble polypeptides. For example, the IL-12 receptor associates with the combination of the p40 polypeptide, similar in structure to soluble forms of hematopoietin receptors such as soluble IL-6R alpha, and the four alpha helix bundle p35 polypeptide. The IL-12 p40 subunit can also associate with another four alpha helix bundle cytokine called p19; when p40 binds p19 the resulting combination has been named “IL-23” and has been shown to bind to the IL-12R beta 1 receptor subunit, but not the signaling IL-12R beta 2 receptor subunit (Oppmann et al., 2000, Immunity 13: 715-725). Thus the p40-p19 complex is likely to bind a different EL-12RB2-like signaling receptor subunit, such as HPR2, HPR1, GCSFR, or gp130. As another example, CNTFR alpha, gp130, and LIFR can each associate with a combination of the soluble receptor cytokine-like factor-1 (CLF-1) and cardiotrophin-like cytokine (CLC), with CLF-1 and CLC analogous to p40 and p35, respectively (Elson et al., 2000, Nat Neurosci 3(9): 867-872). The cytokine receptor domains of HPR1 and HPR2 are similar in sequence to those of gp130, IL-6R beta, IL-12RB2, GCSFR, LIFR, leptin receptor, prolactin receptor, and other members of the hematopoietin receptor family, with HPR1 showing the greatest degree of similarity to gp130 and IL-6R beta, and HPR2 showing the greatest degree of similarity to gp130 and IL-12RB2. Because HPR1 and HPR2 each have a substantial cytoplasmic domain and are most similar in sequence to gp130, HPR1 and HPR2 are likely to be new signaling members of the “gp130-sharing” subfamily of hematopoietin receptors; however, HPR2 may also share attributes of the IL-12RB2 receptor subunit, such as involvement in modulation of the balance between Th1 and Th2 immune responses. Expression of HPR1 and HPR2 has been detected by PCR amplification from tissue-specific cDNA libraries in several cell types including COS-1 cells, 293MSR cells, the B cell lines CB23 and MP-1, the B cell lymphoma lines Daudi, and Raji, the T cell leukemia line HSB2, and the promonocytic leukemia line U937. HPR2 mRNA expression appears to be more prevalent than HPR1 expression in the B cell derived lines, while HPR1 mRNA expression appears to be more prevalent than HPR2 expression in the T cell derived and monocyte lines. EBI-3 is a p40-like soluble hematopoietin receptor polypeptide; FACS analysis has shown that EBI-3-Fc fusion polypeptides bind to cells expressing HPR1 and HPR2 such as COS-1 cells, 293MSR cells, and CB23 and MP-1 cells, indicating that EBI-3 is a potential binding partner of HPR1 and HPR2, most likely in conjunction with a four alpha helix bundle cytokine such as IL-6, OSM, LIF, CNTF, CLC, IL-12p35, or IL-23p19.

Biological Activities and Functions of HPR1 and HPR2 Polypeptides

PCR amplification from tissue-specific cDNA libraries was performed to detect HPR1 or HPR2 cDNA sequences. The results of these experiments show that HPR1 transcripts are expressed in a wide variety of fetal and adult human cells, including testis, lung, placenta, pancreas, prostate, peripheral blood cells, thymus, stomach, and skin cells; as well as in various cell lines including U937 cells, the leukemia cell line HSB2, LX-1/GI-117 lung carcinoma cells, GI-112 colon adenocarcinoma cells, the B cell lines MP-1 and CB23, COS-1 cells, and 293MSR cells. HPR2 transcripts are present in a similarly diverse group of adult and fetal human cell types, including placenta, lung, kidney, pancreas, prostate, testis, colon, LX-1/GI-117 lung carcinoma cells, tonsil/CX-1 cells, lymph node, GI-112 colon adenocarcinoma cells, heart, brain, spleen, thymus, ovary, small intestine, fetal brain, fetal lung/heart, fetal spleen, fetal thymus, esophagus, stomach, and skin; and in various cell lines such as the B cell lines MP-1 and CB23, Daudi cells, Raji cells, HSB2 cells, COS-1 cells, and 293MSR cells.

Typical biological activities or functions associated with HPR1 and HPR2 polypeptides are ligand-binding activity, intracellular signaling activity, cell proliferation stimulatory activity, cell proliferation inhibitory activity, cell differentiation stimulatory activity, and cell differentiation inhibitory activity. HPR1 and HPR2 polypeptides having ligand-binding activity bind to cytokine or growth factor ligand molecules of the four alpha helix bundle family of cytokines, and in particular are likely to bind cytokines such as IL-6, OSM, LWF, CNTF, CLC, IL-12p35, and IL-23p19, and/or soluble hematopoietin receptors such as EBI-3, soluble EL-6R alpha, cytokine-like factor-1 (CLF), IL-12p40, or a soluble form of HPR1 and/or HPR2. This ligand-binding activity is associated with the extracellular cytokine receptor domain of HPR1 polypeptides. Thus, for uses requiring ligand-binding activity, preferred HPR1and HPR2 polypeptides include those having at least one cytokine receptor domain and exhibiting ligand-binding activity. Preferred HPR1 and HPR2 polypeptides further include oligomers or fusion polypeptides comprising at least one cytokine receptor portion of one or more HPR1 and/or HPR2 polypeptides, and fragments of any of these polypeptides that have ligand-binding activity. The ligand-binding activity of HPR1 and HPR2 polypeptides may be determined, for example, by any standard assay to measure binding of labeled ligand or by a competitive binding assay, all of which are described more extensively below. HPR1 and HPR2 polypeptides having intracellular signaling activity bind ligand molecules when in association with other receptor polypeptides to form a homo- or heteromeric complex, with ligand binding initiating a signaling cascade. The intracellular signaling activity is associated with the cytoplasmic domain of certain HPR1 and HPR2 polypeptides. Thus, for uses requiring intracellular signaling activity, preferred HPR1 and HPR2 polypeptides include those having the cytoplasmic domain, and in particular having certain conserved domains (such as the Box 1 motif, the Trp residue at position 581 of SEQ ID NO: 4, the Box 2 motif, the Asp-containing motifs between amino acids 588 and 639 of SEQ ID NO: 4, or the Box 3 motif) and conserved cytoplasmic tyrosine residues, and exhibiting intracellular signaling biological activity. Preferred HPR1 and HPR2 polypeptides further include oligomers or fusion polypeptides comprising at least one cytoplasmic portion of one or more HPR1 and/or HPR2 polypeptides, and fragments of any of these polypeptides that have intracellular signaling activity. The intracellular signaling activity of HPR1 and HPR2 polypeptides may be determined, for example, through assays to detect phosphorylation of the HPR1 polypeptide, the HPR2 polypeptide, or downstream polypeptides in signaling cascades such as the JAK/STAT or ERK/MAPK pathways, or in assays that measure biological activities related to the signal transmission, such as stimulation or suppression of cell proliferation, differentiation, or activation. One example of an assay to measure cytokine-binding and cell-proliferation activity involves expressing a polypeptide of the invention in Ba/F3 cells, exposing the polypeptide-expressing cells to radioactively labeled cytokine, and measuring specific cytokine binding to cells and uptake of 3H-thymidine by cells in response to cytokine, as described in Presky et al., 1996, Proc Natl Acad Sci USA 93: 14002-14007. Further examples of such assays are described herein and in Ernst et al., 1999, J Biol Chem 274(14): 9729-9737. Soluble forms of hematopoietin receptors comprising one or more extracellular domains of the hematopoietin receptor, such as soluble forms of HPR1 and HPR2, may also be used in assays to measure their effect on cell growth, proliferation, differentiation, or activation; in such assays the cells are contacted with the soluble form of the receptor and their growth, proliferation, differentiation, or activation is measured, for example by measuring the incorporation of radioactive thymidine or by microscopic examination of treated and untreated cells.

The terms “HPR1 polypeptide activity” and “HPR2 polypeptide activity,” as used herein, include any one or more of the following: ligand-binding activity and intracellular signaling activity (which includes effects on cell growth, proliferation, differentiation, or activation), as well as the ex vivo and in vivo activities of HPR1 and HPR2 polypeptides. The degree to which HPR1 and HPR2 polypeptides and fragments and other derivatives of these polypeptides exhibit these activities can be determined by standard assay methods as disclosed herein; those of skill in the art will appreciate that other, similar types of assays can be used to measure HPR1 and HPR2 biological activities.

Another aspect of the biological activity of HPR1 and HPR2 polypeptides is the ability of members of these polypeptide families to bind particular binding partners such as cytokines, other hematopoietin receptor polypeptides, and intracellular signaling polypeptides, with the cytokine receptor domain binding to cytokines and the intracellular signaling domain binding to intracellular signaling polypeptides such as members of the JAK and SHP polypeptide families. The term “binding partner,” as used herein, includes ligands, receptors, substrates, antibodies, other hematopoietin receptor polypeptides, the same HPR1 or HPR2 polypeptide (in the case of homotypic interactions), and any other molecule that interacts with an HPR1 or an HPR2 polypeptide through contact or proximity between particular portions of the binding partner and the HPR1 or HPR2 polypeptide. Because the cytokine receptor domains of HPR1 and HPR2 polypeptides bind to cytokines, an HPR1 or HPR2 cytokine receptor domain when expressed as a separate fragment from the rest of an HPR1 or HPR2 polypeptide, or as a soluble polypeptide, fused for example to an immunoglobulin Fc domain, is expected to disrupt the binding of endogenous HPR1 and/or HPR2 polypeptides to their binding partners. By binding to one or more binding partners, the separate cytokine receptor domain polypeptide likely prevents binding by the native HPR1 and/or HPR2 polypeptide(s), and so acts in a dominant negative fashion to inhibit the biological activities mediated via binding of HPR1 and/or HPR2 polypeptides to cytokines. Assays for evaluating the biological activities and partner-binding properties of HPR1 and HPR2 polypeptides are described further herein.

HPR1 and HPR2 polypeptides are involved in cell proliferation, differentiation, or activation diseases or conditions, that share as a common feature ligand-binding activity in their etiology. More specifically, the following cell proliferation conditions are those that are known or are likely to involve the biological activities of HPR1 and/or HPR2 polypeptides: pancytopenia, leukopenia, anemia, thrombocytopenia, neurodegenerative disorders, osteoporosis resulting from a lack of bone-forming cells, leukemia, tumour metastasis, and osteoporosis resulting from an excess of bone-resorbing cells. In addition, the following metabolic conditions involving hematopoietin receptor ligands such as leptin are those that are known or are likely to involve the biological activities of HPR1 and/or HPR2 polypeptides: obesity, cachexia, wasting, and AIDS-related weight loss. Also, the following prolactin-related conditions are those that are known or are likely to involve the biological activities of HPR1 and/or HPR2 polypeptides: deficient mammary development, infertility, breast cancer, and prolactinoma. Blocking or inhibiting the interactions between members of the HPR1 and HPR2 polypeptide families and their substrates, ligands, receptors, binding partners, and or other interacting polypeptides is an aspect of the invention and provides methods for treating or ameliorating these diseases and conditions through the use of inhibitors of HPR1 and/or HPR2 polypeptide activity. Examples of such inhibitors or antagonists are described in more detail below. For certain conditions involving too little HPR1 or HPR2 polypeptide activity, methods of treating or ameliorating these conditions comprise increasing the amount or activity of HPR1 or HPR2 polypeptides by providing isolated HPR1 or HPR2 polypeptides or active fragments or fusion polypeptides thereof, or by providing compounds (agonists) that activate endogenous or exogenous HPR1 or HPR2 polypeptides.

HPR1 and HPR2 Polypeptides

An HPR1 polypeptide is a polypeptide that shares a sufficient degree of amino acid identity or similarity to the human HPR1 polypeptide of SEQ ID NO: 4 or the murine HPR1 polypeptide of SEQ ID NO: 12 to (A) be identified by those of skill in the art as a polypeptide likely to share particular structural domains and/or (B) have biological activities in common with the HPR1 polypeptides of SEQ ID NO: 4 and SEQ ID NO: 12 and/or (C) bind to antibodies that also specifically bind to other HPR1 polypeptides. An HPR2 polypeptide is a polypeptide that shares a sufficient degree of amino acid identity or similarity to the HPR2 polypeptides of SEQ ID NOs 21, 23, 25, and 27 to (A) be identified by those of skill in the art as a polypeptide likely to share particular structural domains and/or (B) have biological activities in common with the HPR2 polypeptides of SEQ ID NOs 21, 23, 25, and 27 and/or (C) bind to antibodies that also specifically bind to other HPR2 polypeptides. HPR1 and HPR2 polypeptides can be isolated from naturally occurring sources, or have the same structure as naturally occurring HPR1 or HPR2 polypeptides, or can be produced to have structures that differ from naturally occurring HPR1 or HPR2 polypeptides. Polypeptides derived from any HPR1 or HPR2 polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides or molecules) are also HPR1 or HPR2 polypeptides, respectively. Therefore, the polypeptides provided by the invention include polypeptides characterized by amino acid sequences similar to those of the HPR1 and HPR2 polypeptides described herein, but into which modifications are naturally provided or deliberately engineered. A polypeptide that shares biological activities in common with members of the HPR1 and/or HPR2 polypeptide family is a polypeptide having HPR1 and/or HPR2 polypeptide activity. Examples of biological activities exhibited by HPR1 and/or HPR2 polypeptides include, without limitation, ligand-binding activity and intracellular signaling.

The present invention provides both full-length and mature forms of HPR1 and HPR2 polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide can be obtained by expression, in a suitable mammalian cell or other host cell, of a nucleic acid molecule that encodes the full-length polypeptide. The sequence of the mature form of the polypeptide may also be determinable from the amino acid sequence of the full-length form, through identification of signal sequences or protease cleavage sites. The HPR1 and HPR2 polypeptides of the invention also include those that result from post-transcriptional or post-translational processing events such as alternate mRNA processing which can yield alternative splice forms of HPR1 or HPR2 such as a truncated but biologically active polypeptide or, for example, a naturally occurring soluble form of the polypeptide. Also encompassed within the invention are variations attributable to proteolysis such as differences in the N- or C-termini upon expression in different types of host cells, due to proteolytic removal of one or more terminal amino acids from the polypeptide (generally from 1-5 terminal amino acids).

The invention further includes HPR1 and HPR2 polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of HPR1 and HPR2 polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of HPR1 and HPR2 polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the HPR1 and HPR2 polypeptides of the present invention are encompassed by the present invention and can be in linear form or cyclized using known methods, for example, as described in Saragovi, et al., Bio/Technology 10, 773-778 (1992) and in McDowell, et al., J. Amer. Chem. Soc. 114 9245-9253 (1992). Polypeptides and polypeptide fragments of the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of an HPR1 polypeptide or of an HPR2 polypeptide, and have at least 60% sequence identity (more preferably at least 70%, at least 75%, at least 80%, at least 85%, at least 90%, at least 95%, at least 97.5%, or at least 99%, and most preferably at least 99.5%) with that HPR1 or HPR2 polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any of the HPR1 or HPR2 polypeptides, where sequence identity is determined by comparing the amino acid sequences of the polypeptides when aligned so as to maximize overlap and identity while minimizing sequence gaps. The percent identity can be determined by visual inspection and mathematical calculation. Preferably, the comparison is done using a computer program. An exemplary, preferred computer program is the Genetics Computer Group (GCG; Madison, Wis.) Wisconsin package version 10.0 program, ‘GAP.’ The preferred default parameters for the ‘GAP’ program includes: (1) The GCG implementation of comparison matrices for nucleotides and amino acids; such as a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Another program useful for determining percent identify is the BESTFIT program, also available from the University of Wisconsin as part of the GCG computer package. Default parameters for using the BESTFIT program are the same as those described above for using the GAP program. Other programs used by those skilled in the art of sequence comparison can also be used, such as, for example, the UW-BLAST 2.0 algorithm or the BLASTN program version 2.0.9, available for use via the National Library of Medicine website: ncbi.nlm.nih.gov/gorf/wblast2.cgi. Standard default parameter settings for UW-BLAST 2.0 are described at the following Internet site: blast.wustl.edu/blast/README.html#References. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

“An isolated polypeptide consisting essentially of an amino acid sequence” means that the polypeptide may have, in addition to said amino acid sequence, additional material covalently linked to either or both ends of the polypeptide, said additional material preferably between 1 and 10,000 additional amino acids covalently linked to either end, each end, or both ends of polypeptide, and more preferably between 1 and 1,000 additional amino acids covalently linked to either end, each end, or both ends of the polypeptide, and most preferably between 1 and 100 additional amino acids covalently linked to either end, each end, or both ends of the polypeptide. In preferred embodiments, covalent linkage of additional amino acids to either end, each end, or both ends of the polypeptide results in a novel combined amino acid sequence that is neither naturally occurring nor disclosed in the art.

The present invention also provides for soluble forms of HPR1 and HPR2 polypeptides comprising or consisting essentially of certain fragments or domains of these polypeptides, and particularly those comprising the extracellular domain or one or more fragments of the extracellular domain. Soluble polypeptides are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble HPR1 and HPR2 polypeptides also include those polypeptides which include part of the transmembrane region, provided that the soluble HPR1 or HPR2 polypeptide is capable of being secreted from a cell, and preferably retains HPR1 and/or HPR2 polypeptide activity. Soluble HPR1 and HPR2 polypeptides further include oligomers or fusion polypeptides comprising the extracellular portion of at least one HPR1 or HPR2 polypeptide, and fragments of any of these polypeptides that have HPR1 and/or HPR2 polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of HPR1 or HPR2 polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect of the invention, preferred polypeptides comprise various combinations of HPR1 and/or HPR2 polypeptide domains, such as the cytokine receptor domain and the intracellular signaling domain. Accordingly, polypeptides of the present invention and nucleic acids encoding them include those comprising or encoding two or more copies of a domain such as the cytokine receptor domain, two or more copies of a domain such as the intracellular signaling domain, or at least one copy of each domain, and these domains can be presented in any order within such polypeptides.

Further modifications in the peptide or DNA sequences can be made by those skilled in the art using known techniques. Modifications of interest in the polypeptide sequences can include the alteration, substitution, replacement, insertion or deletion of a selected amino acid. For example, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. fI-B 9259. Monoclonal antibodies that bind the FLAG® peptide are available from Eastman Kodak Co., Scientific Imaging Systems Division, New Haven, Connecticut.

Encompassed by the invention are oligomers or fusion polypeptides that contain an HPR1 polypeptide and/or an HPR2 polypeptide, one or more fragments of HPR1 and/or HPR2 polypeptides, or any of the derivative or variant forms of HPR1 and HPR2 polypeptides as disclosed herein. In particular embodiments, the oligomers comprise soluble HPR1 and/or HPR2 polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple HPR1 and/or HPR2 polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

In embodiments where variants of the HPR1 and/or HPR2 polypeptides are constructed to include a membrane-spanning domain, they will form a Type I membrane polypeptide. Membrane-spanning HPR1 and/or HPR2 polypeptides can be fused with extracellular domains of receptor polypeptides for which the ligand is known. Such fusion polypeptides can then be manipulated to control the intracellular signaling pathways triggered by the membrane-spanning HPR1 or HPR2 polypeptide. HPR1 and HPR2 polypeptides that span the cell membrane can also be fused with agonists or antagonists of cell-surface receptors, or cellular adhesion molecules to further modulate HPR1 and/or HPR2 intracellular effects. In another aspect of the present invention, interleukins can be situated between the preferred HPR1 or HPR2 polypeptide fragment and other fusion polypeptide domains.

Immunoglobulin-based Oligomers. The polypeptides of the invention or fragments thereof can be fused to molecules such as immunoglobulins for many purposes, including increasing the valency of polypeptide binding sites. For example, fragments of an HPR1 polypeptide and/or of an HPR2 polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgGI antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four HPR1 and/or HPR2 extracellular regions.

Peptide-linker Based Oligomers. Alternatively, the oligomer is a fusion polypeptide comprising multiple HPR1 and/or HPR2 polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble HPR1 and/or HPR2 polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art.

Other fragments and derivatives of the sequences of polypeptides which would be expected to retain polypeptide activity in whole or in part and may thus be useful for screening or other immunological methodologies can also be made by those skilled in the art given the disclosures herein. Such modifications are believed to be encompassed by the present invention.

Nucleic Acids Encoding HPR1 Polypeptides and Nucleic Acids Encoding HPR2 Polypeptides

Encompassed within the invention are nucleic acids encoding HPR1 polypeptides and nucleic acids encoding HPR2 polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a DNA sequence encoding an HPR1 or HPR2 polypeptide or a desired combination of HPR1 and/or HPR2 polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the invention relates to certain isolated nucleic acids that are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

“An isolated nucleic acid consisting essentially of a nucleotide sequence” means that the nucleic acid may have, in addition to said nucleotide sequence, additional material covalently linked to either or both ends of the nucleic acid molecule, said additional material preferably between 1 and 100,000 additional nucleotides covalently linked to either end, each end, or both ends of the nucleic acid molecule, and more preferably between 1 and 1,000 additional nucleotides covalently linked to either end, each end, or both ends of the nucleic acid molecule, and most preferably between 10 and 100 additional nucleotides covalently linked to either end, each end, or both ends of the nucleic acid molecule. In preferred embodiments, covalent linkage of additional nucleotides to either end, each end, or both ends of the nucleic acid molecule results in a novel combined nucleotide sequence that is neither naturally occurring nor disclosed in the art. An isolated nucleic acid consisting essentially of a nucleotide sequence may be an expression vector or other construct comprising said nucleotide sequence.

The present invention also includes nucleic acids that hybridize under moderately stringent conditions, and more preferably highly stringent conditions, to nucleic acids encoding HPR1 polypeptides and/or nucleic acids encoding HPR2 polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5×SSC, 0.1% SDS. Generally, highly stringent conditions are defined as hybridization conditions as above, but with washing at approximately 68 degrees C., 0.2×SSC, 0.1% SDS. SSPE (1×SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10.degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀ [Na+])+0.41(% G+C)−(600/N), where N is the number of bases in the hybrid, and [Na⁺] is the concentration of sodium ions in the hybridization buffer ([Na⁺] for 1×SSC=0. 165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (more preferably, 5000 basepairs, still more preferably, 2500 basepairs, and most preferably, 1000 basepairs) of genomic nucleic acid sequence upstream of the first nucleotide of the genomic sequence corresponding to the initiation codon of the HPR1 coding sequence or of the HPR2 coding sequence, and 10000 basepairs (more preferably, 5000 basepairs, still more preferably, 2500 basepairs, and most preferably, 1000 basepairs) of genomic nucleic acid sequence downstream of the last nucleotide of the genomic sequence corresponding to the termination codon of the HPR1 coding sequence or of the HPR2 coding sequence. The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or an “isolated genomic nucleic acid” is a genomic nucleic acid that has been separated from the adjacent genomic sequences present in the genome of the organism from which the genomic nucleic acid was isolated.

Methods for Making and Purifying HPR1 and HPR2 Polypeptides

Methods for making HPR1 and HPR2 polypeptides are described below. Expression, isolation, and purification of the polypeptides and fragments of the invention can be accomplished by any suitable technique, including but not limited to the following methods. The isolated nucleic acid of the invention can be operably linked to an expression control sequence such as the pDC409 vector (Giri et al., 1990, EMBO J., 13: 2821) or the derivative pDC412 vector (Wiley et al., 1995, Immunity 3: 673). The pDC400 series vectors are useful for transient mammalian expression systems, such as CV-1 or 293 cells. Alternatively, the isolated nucleic acid of the invention can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Other expression control sequences and cloning technologies can also be used to produce the polypeptide recombinantly, such as the pMT2 or pED expression vectors (Kaufman et al., 1991, Nucleic Acids Res. 19: 4485-4490; and Pouwels et al., 1985, Cloning Vectors: A Laboratory Manual, Elsevier, N.Y.) and the GATEWAY Vectors (lifetech.com/Content/Tech-Online/molecular_biology/manuals_pps/11797016.pdf; Life Technologies; Rockville, Md.). In the GATEWAY system the isolated nucleic acid of the invention, flanked by attb sequences, can be recombined through an integrase reaction with a GATEWAY vector such as pDONR201 containing attP sequences. This provides an entry vector for the GATEWAY system containing the isolated nucleic acid of the invention. This entry vector can be further recombined with other suitably prepared expression control sequences, such as those of the pDC400 and pDC300 series described above. Many suitable expression control sequences are known in the art. General methods of expressing recombinant polypeptides are also known and are exemplified in R. Kaufman, Methods in Enzymology 185, 537-566 (1990). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells promotes extracellular secretion of the polypeptide. The signal peptide is cleaved from the polypeptide upon secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable strain for DHFR selection can be CHO strain DX-B 11, which is deficient in DBFR (Urlaub and Chasin, Proc. Natl. Acad. Sci. USA 77:4216-4220, 1980). A plasmid expressing the DHFR cDNA can be introduced into strain DX-B 11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells can act as suitable host cells for expression of the polypeptide. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al. (EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it is possible to produce the polypeptide in lower eukaryotes such as yeast or in prokaryotes such as bacteria. Potentially suitable yeasts include Saccharomyces cerevisiae, Schizosaccharomyces pombe, Kluyveromyces strains, Candida, or any yeast strain capable of expressing heterologous polypeptides. Potentially suitable bacterial strains include Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments can be accomplished using known chemical or enzymatic methods. The polypeptide can also be produced by operably linking the isolated nucleic acid of the invention to suitable control sequences in one or more insect expression vectors, and employing an insect expression system. Materials and methods for baculoviruslinsect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). As used herein, an insect cell capable of expressing a nucleic acid of the present invention is “transformed.” Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”.

The polypeptide of the invention can be prepared by culturing transformed host cells under culture conditions suitable to express the recombinant polypeptide. The resulting expressed polypeptide can then be purified from such culture (i.e., from culture medium or cell extracts) using known purification processes, such as gel filtration and ion exchange chromatography. The purification of the polypeptide can also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the invention can also be expressed in a form which will facilitate purification. For example, it can be expressed as a fusion polypeptide, such as those of maltose binding polypeptide (MBP), glutathione-S-transferase (GST) or thioredoxin (TRX). Kits for expression and purification of such fusion polypeptides are commercially available from New England BioLab (Beverly, Mass.), Pharmacia (Piscataway, N.J.) and InVitrogen, respectively. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to HPR1 and/or HPR2 polypeptides, fragments, variants, binding partners etc. The polypeptide of the invention can also be expressed as a product of transgenic animals, e.g., as a component of the milk of transgenic cows, goats, pigs, or sheep which are characterized by somatic or germ cells containing a nucleotide sequence encoding the polypeptide.

It is also possible to utilize an affinity column comprising a polypeptide-binding polypeptide of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention. In this aspect of the invention, polypeptide-binding polypeptides, such as the anti-polypeptide antibodies of the invention or other polypeptides that can interact with the polypeptide of the invention, can be bound to a solid phase support such as a column chromatography matrix or a similar substrate suitable for identifying, separating, or purifying cells that express polypeptides of the invention on their surface. Adherence of polypeptide-binding polypeptides of the invention to a solid phase contacting surface can be accomplished by any means, for example, magnetic microspheres can be coated with these polypeptide-binding polypeptides and held in the incubation vessel through a magnetic field. Suspensions of cell mixtures are contacted with the solid phase that has such polypeptide-binding polypeptides thereon. Cells having polypeptides of the invention on their surface bind to the fixed polypeptide-binding polypeptide and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. Alternatively, mixtures of cells suspected of containing polypeptide-expressing cells of the invention first can be incubated with a biotinylated polypeptide-binding polypeptide of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The polypeptide can also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with HPR1 and/or HPR2 polypeptides can possess biological properties in common therewith, including HPR1 and/or HPR2 polypeptide activity. Thus, they can be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Antagonists and Agonists of HPR1 and/or HPR2 Polypeptides

Any method which neutralizes HPR1 and/or HPR2 polypeptides or inhibits expression of the HPR1 and/or HPR2 genes (either transcription or translation) can be used to reduce the biological activities of HPR1 and/or HPR2 polypeptides. In particular embodiments, antagonists inhibit the binding of at least one HPR1 polypeptide and/or at least one HPR2 polypeptide to cells, thereby inhibiting biological activities induced by the binding of those HPR1 or HPR2 polypeptides to the cells. In certain other embodiments of the invention, antagonists can be designed to reduce the level of endogenous HPR1 and/or HPR2 gene expression, e.g., using well-known antisense or ribozyme approaches to inhibit or prevent translation of HPR1 and/or HPR2 mRNA transcripts; triple helix approaches to inhibit transcription of HPR1 and/or HPR2 genes; or targeted homologous recombination to inactivate or “knock out” the HPR1 gene(s), the HPR2 gene(s), or their endogenous promoters or enhancer elements. Such antisense, ribozyme, and triple helix antagonists can be designed to reduce or inhibit either unimpaired, or if appropriate, mutant HPR1 and/or HPR2 gene activity. Techniques for the production and use of such molecules are well known to those of skill in the art.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to an HPR1 and/or to an HPR2 mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides are complementary to the 5′ end of the message, e.g., the 5′ untranslated sequence up to and including the AUG initiation codon. However, oligonucleotides complementary to the 5′- or 3′-non-translated, non-coding regions of the HPR1 or HPR2 gene transcript(s) could be used in an antisense approach to inhibit translation of endogenous HPR1 and/or HPR2 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleotides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci USA 86:6553-6556; Lemaitre et al., 1987, Proc Natl Acad Sci 84:648-652; PCT Publication No. WO88/09810), or hybridization-triggered cleavage agents or intercalating agents. (See, e.g., Zon, 1988, Pharm. Res. 5:539-549). The antisense molecules should be delivered to cells which express the HPR1 and/or HPR2 transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous HPR1 and/or HPR2 gene transcripts and thereby prevent translation of the HPR1 and/or HPR2 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave HPR1 and/or HPR2 mRNA transcripts can also be used to prevent translation of HPR1 and/or HPR2 mRNA and expression of HPR1 and/or HPR2 polypeptides. (See, e.g., PCT International Publication WO90/11364 and U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express HPR1 and/or HPR2 polypeptides in vivo. A preferred method of delivery involves using a DNA construct “encoding” the ribozyme under the control of a strong constitutive pol III or pol II promoter, so that transfected cells will produce sufficient quantities of the ribozyme to destroy endogenous HPR1 and/or HPR2 messages and inhibit translation. Because ribozymes, unlike antisense molecules, are catalytic, a lower intracellular concentration is required for efficiency.

Alternatively, endogenous HPR1 and/or HPR2 gene expression can be reduced by targeting deoxyribonucleotide sequences complementary to the regulatory region of the target gene (i.e., the target gene promoter and/or enhancers) to form triple helical structures that prevent transcription of the target HPR1 and/or HPR2 gene. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Endogenous target gene expression can also be reduced by inactivating or “knocking out” the target gene or its promoter using targeted homologous recombination (e.g., see Smithies, et al., 1985, Nature 317, 230-234; Thomas and Capecchi, 1987, Cell 51, 503-512; Thompson, et al., 1989, Cell 5, 313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok, Tabara, and Mello, 2000, Genetic requirements for inheritance of RNAi in C. elegans, Science 287 (5462): 2494-2497), or the introduction of transgenes (Dernburg et al., 2000, Transgene-mediated cosuppression in the C. elegans germ line, Genes Dev. 14 (13): 1578-1583) are used to inhibit the expression of specific target genes. However this approach can be adapted for use in humans provided the recombinant DNA constructs are directly administered or targeted to the required site in vivo using appropriate vectors such as viral vectors.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc Natl Acad Sci USA 90(16): 7431-7435; Clark et al., 1994, Proc Natl Acad Sci USA 91(2): 719-722), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are HPR1 and HPR2 polypeptide variants with partner binding sites that have been altered in conformation so that (1) the HPR1 or HPR2 variant will still bind to its partner(s), but a specified small molecule will fit into the altered binding site and block that interaction, or (2) the HPR1 or HPR2 variant will no longer bind to its partner(s) unless a specified small molecule is present (see for example Bishop et al., 2000, Nature 407: 395-401). Nucleic acids encoding such altered HPR1 or HPR2 polypeptides can be introduced into organisms according to methods described herein, and can replace the endogenous nucleic acid sequences encoding the corresponding HPR1 or HPR2 polypeptide. Such methods allow for the interaction of a particular HPR1 or HPR2 polypeptide with its binding partners to be regulated by administration of a small molecule compound to an organism, either systemically or in a localized manner.

The HPR1 and HPR2 polypeptides themselves can also be employed in inhibiting a biological activity of HPR1 and /or of HPR2 in in vitro or in vivo procedures. Encompassed within the invention are cytokine receptor domains of HPR1 and HPR2 polypeptides that act as “dominant negative” inhibitors of native HPR1 and/or HPR2 polypeptide function when expressed as fragments or as components of fusion polypeptides. For example, a purified polypeptide domain of the present invention can be used to inhibit binding of HPR1 or HPR2 polypeptides to endogenous binding partners. Such use effectively would block HPR1 and/or HPR2 polypeptide interactions and inhibit HPR1 and/or HPR2 polypeptide activities. In still another aspect of the invention, a soluble form of an HPR1 and/or HPR2 binding partner is used to bind to an endogenous HPR1 and/or HPR2 polypeptide, and competitively inhibit activation of that endogenous HPR1 and/or HPR2 polypeptide. Furthermore, antibodies which bind to HPR1 and/or HPR2 polypeptides often inhibit HPR1 and/or HPR2 polypeptide activity and act as antagonists. For example, antibodies that specifically recognize one or more epitopes of HPR1 and/or HPR2 polypeptides, or epitopes of conserved variants of HPR1 and/or HPR2 polypeptides, or peptide fragments of an HPR1 and/or HPR2 polypeptide can be used in the invention to inhibit HPR1 and/or HPR2 polypeptide activity. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified HPR1 and/or HPR2 polypeptides of the present invention can be administered to modulate interactions between HPR1 and/or HPR2 polypeptides and HPR1 and/or HPR2 binding partners that are not membrane-bound. Such an approach will allow an alternative method for the modification of HPR1- and/or HPR2-influenced bioactivity.

In an alternative aspect, the invention further encompasses the use of agonists of HPR1 and/or HPR2 polypeptide activity to treat or ameliorate the symptoms of a disease for which increased HPR1 and/or HPR2 polypeptide activity is beneficial. Such diseases include but are not limited to pancytopenia, leukopenia, anemia, thrombocytopenia, neurodegenerative disorders, osteoporosis resulting from a lack of bone-forming cells, obesity, deficient mammary development, and infertility. In a preferred aspect, the invention entails administering compositions comprising an HPR1 or HPR2 nucleic acid or an HPR1 or HPR2 polypeptide to cells in vitro, to cells ex vivo, to cells in vivo, and/or to a multicellular organism such as a vertebrate or mammal. Preferred therapeutic forms of HPR1 and HPR2 are soluble forms, as described above. In still another aspect of the invention, the compositions comprise administering an HPR1-encoding nucleic acid or an HPR2-encoding nucleic acid for expression of an HPR1 or HPR2 polypeptide in a host organism for treatment of disease. Particularly preferred in this regard is expression in a human patient for treatment of a dysfunction associated with aberrant (e.g., decreased) endogenous activity of an HPR1 or HPR2 polypeptide. Furthermore, the invention encompasses the administration to cells and/or organisms of compounds found to increase the endogenous activity of HPR1 and/or HPR2 polypeptides. One example of compounds that increase HPR1 and/or HPR2 polypeptide activity are agonistic antibodies, preferably monoclonal antibodies, that bind to HPR1 and/or HPR2 polypeptides or binding partners, which may increase HPR1 and/or HPR2 polypeptide activity by causing constitutive intracellular signaling (or “ligand mimicking”), or by preventing the binding of a native inhibitor of HPR1 and/or HPR2 polypeptide activity.

Antibodies to HPR1 and/or HPR2 Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with HPR1 and/or HPR2 polypeptides, homologues, and variants, but not with other molecules. In one preferred embodiment, the antibodies are specific for the polypeptides of the present invention and do not cross-react with other polypeptides. In this manner, the HPR1 and HPR2 polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hinderances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kosbor et al., 1984, J Immunol 133: 3001-3005; Cole et al., 1983, Proc Natl Acad Sci USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: an HPR1 or HPR2 polypeptide, a fragment of an HPR1 or HPR2 polypeptide, a functional equivalent of an HPR1 or HPR2 polypeptide, or a mutant form of an HPR1 or HPR2 polypeptide. Such host animals can include but are not limited to rabbits, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314: 452-454; Morrison et al., 1984, Proc Natl Acad Sci USA 81: 6851-6855; Boulianne et al., 1984, Nature 312: 643-646; Neuberger et al., 1985, Nature 314: 268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat Biotechnol. 16(6): 535-539; and U.S. Pat. No. 5,969,108).

Antigen-binding antibody fragments which recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against HPR1 and/or HPR2 gene products. Single chain antibodies are formed by linking the heavy and light chain fragments of the Fv region via an amino acid bridge, resulting in a single chain polypeptide. Such single chain antibodies can also be useful intracellularly (i.e., as ‘intrabodies), for example as described by Marasco et al. (J. Immunol. Methods 231:223-238, 1999) for genetic therapy in HIV infection. In addition, antibodies to the HPR1 and/or HPR2 polypeptide can, in turn, be utilized to generate anti-idiotype antibodies that “mimic” the HPR1 and/or HPR2 polypeptide and that may bind to the binding partner(s) of HPR1 and/or HPR2 polypeptides, using techniques well known to those skilled in the art. (See, e.g., Greenspan & Bona, 1993, FASEB J 7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9: 635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305: 537-540; U.S. Pat. No. 4,474,893; and U.S. Pat. No. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229: 81-83; Glennie et al., J. Immunol., 1987, 139:2367-2375; and U.S. Pat. No. 6,010,902). Bispecific antibodies can also be produced via recombinant means, for example, by using. the leucine zipper moieties from the Fos and Jun proteins (which preferentially form heterodimers) as described by Kostelny et al. (J. Immnol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CHI domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface HPR1 and/or HPR2, induce biological effects (e.g., transduction of biological signals) similar to the biological effects induced when the HPR1 and/or HPR2 binding partner binds to cell surface HPR1 and/or HPR2. Agonistic antibodies can be used to induce HPR1- and/or HPR2-mediated intracellular signaling or cell proliferation. Bispecific antibodies can be identified by screening with two separate assays, or with an assay wherein the bispecific antibody serves as a bridge between the first antigen and the second antigen (the latter is coupled to a detectable moiety). Bispecific antibodies that bind HPR1 and/or HPR2 polypeptides of the invention via a first antigen binding domain will be useful in diagnostic applications and in treating cell proliferation, differentiation, or activation diseases or conditions. Examples of polypeptides (or other antigens) that the inventive bispecific antibodies bind via a second antigen binding domain include: four alpha helix bundle cytokines such as EL-6, OSM, LIF, CNTF, CLC, L-12p35, and IL-23p19; soluble hematopoietin receptors such as EBI-3, soluble IL-6R alpha, cytokine-like factor-1 (CLF), IL-12p40, or a soluble form of HPR1 and/or HPR2; and soluble hematopoietin receptors such as EBI-3 etc. in conjunction with a four alpha helix bundle cytokine.

Those antibodies that can block binding of the HPR1 and/or HPR2 polypeptides of the invention to binding partners for HPR1 and/or HPR2 can be used to inhibit HPR1- and/or HPR2-mediated intracellular signaling or cell proliferation that results from such binding. Such blocking antibodies can be identified using any suitable assay procedure, such as by testing antibodies for the ability to inhibit binding of HPR1 and/or HPR2 to certain cells expressing an HPR1 and/or HPR2 binding partner. Alternatively, blocking antibodies can be identified in assays for the ability to inhibit a biological effect that results from binding of soluble HPR1 and/or HPR2 to target cells. Antibodies can be assayed for the ability to inhibit HPR1 and/or HPR2 binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of HPR1 and/or HPR2 with cell surface binding partner receptor thus can be treated. A therapeutic method involves in vivo administration of a blocking antibody to a mammal in an amount effective in inhibiting HPR1 and/or HPR2 binding partner-mediated biological activity. Monoclonal antibodies are generally preferred for use in such therapeutic methods. In one embodiment, an antigen-binding antibody fragment is employed. Compositions comprising an antibody that is directed against HPR1 and/or HPR2, and a physiologically acceptable diluent, excipient, or carrier, are provided herein. Suitable components of such compositions are as described below for compositions containing HPR1 and/or HPR2 polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides or fragments of the invention, either in vitro or in vivo. The antibodies also can be employed in purifying polypeptides or fragments of the invention by immunoaffinity chromatography.

Rational Design of Compounds that Interact with HPR1 and/or HPR2 Polypeptides

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the polypeptide or which enhance or interfere with the function of a polypeptide in vivo (Hodgson J (1991) Biotechnology 9:19-21). In one approach, the three-dimensional structure of a polypeptide of interest, or of a polypeptide-inhibitor complex, is determined by x-ray crystallography, by nuclear magnetic resonance, or by computer homology modeling or, most typically, by a combination of these approaches. Both the shape and charges of the polypeptide must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous HPR1- and/or HPR2-like molecules, to identify efficient inhibitors, or to identify small molecules that bind HPR1 and/or HPR2 polypeptides. Useful examples of rational drug design include molecules which have improved activity or stability as shown by Braxton S and Wells J A (1992 Biochemistry 31:7796-7801) or which act as inhibitors, agonists, or antagonists of native peptides as shown by Athauda S B et al (1993 J Biochem 113:742-746). The use of HPR1 and/or HPR2 polypeptide structural information in molecular modeling software systems to assist in inhibitor design and in studying inhibitor-HPR1 polypeptide and/or inhibitor-HPR2 polypeptide interaction is also encompassed by the invention. A particular method of the invention comprises analyzing the three-dimensional structure of HPR1 and/or HPR2 polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

Assays of HPR1 and HPR2 Polypeptide Activities

The purified HPR1 and HPR2 polypeptides of the invention (including polypeptides, polypeptides, fragments, variants, oligomers, and other forms) are useful in a variety of assays. For example, the HPR1 and HPR2 molecules of the present invention can be used to identify binding partners of HPR1 and/or HPR2 polypeptides, which can also be used to modulate intracellular signaling, cell proliferation, or immune cell activity. Alternatively, they can be used to identify non-binding-partner molecules or substances that modulate intracellular signaling, cell proliferation, or immune cell activity.

Assays to Identify Binding Partners. HPR1 and HPR2 polypeptides and fragments thereof can be used to identify binding partners. For example, they can be tested for the ability to bind a candidate binding partner in any suitable assay, such as a conventional binding assay. To illustrate, the HPR1 or HPR2 polypeptide can be labeled with a detectable reagent (e.g., a radionuclide, chromophore, enzyme that catalyzes a colorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al., (EMBO J. 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82: 8672-8676), homogeneous time-resolved fluorescence methods (Park et al., 1999, Anal Biochem 269: 94-104), fluorescence resonance energy transfer (FRET) methods (Clegg RM, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden). Compounds that can be assayed for binding to HPR1 and/or HPR2 polypeptides include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co.(Trevillett, Cornwall, UK), MDS Panlabs (Bothell, Wash.), Pharmacopeia (Princeton, N.J.), and Trega (San Diego, Calif.). Preferred small organic molecules for screening using these assays are usually less than 10 K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2): 193-198). Compounds including natural products, inorganic chemicals, and biologically active materials such as proteins and toxins can also be assayed using these methods for the ability to bind to HPR1 and/or HPR2 polypeptides.

Yeast Two-Hybrid or “Interaction Trap” Assays. Because HPR1 and HPR2 polypeptides bind or potentially bind to another polypeptide (such as, for example, in a receptor-ligand interaction), the nucleic acid encoding the HPR1 or HPR2 polypeptide can also be used in interaction trap assays (such as, for example, that described in Gyuris et al., Cell 75:791-803 (1993)) to identify nucleic acids encoding the other polypeptide with which binding occurs, or to identify inhibitors of the binding interaction. Polypeptides involved in these binding interactions can also be used to screen for peptide or small molecule inhibitors or agonists of the binding interaction.

Competitive Binding Assays. Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled HPR1 or HPR2 and intact cells expressing HPR1 and/or HPR2 (endogenous or recombinant) on the cell surface. For example, a radiolabeled soluble HPR1 or HPR2 fragment can be used to compete with a soluble HPR1 variant and/or a soluble HPR2 variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Assays to Identify Modulators of Intracellular Signaling, Cell Proliferation, or Immune Cell Activity. The influence of HPR1 or HPR2 on intracellular signaling, cell proliferation, or immune cell activity can be manipulated to control these activities in target cells. For example, the disclosed HPR1 and HPR2 polypeptides, nucleic acids encoding the disclosed HPR1 and HPR2 polypeptides, or agonists or antagonists of such polypeptides can be administered to a cell or group of cells to induce, enhance, suppress, or arrest intracellular signaling or cell proliferation by the target cells. Identification of HPR1 and HPR2 polypeptides, agonists or antagonists that can be used in this manner can be carried out via a variety of assays known to those skilled in the art. Included in such assays are those that evaluate the ability of an HPR1 or HPR2 polypeptide to influence intracellular signaling, cell proliferation, or immune cell activity. Such an assay would involve, for example, the analysis of immune cell interaction in the presence of an HPR1 polypeptide and/or an HPR1 polypeptide. In such an assay, one would determine a rate of intracellular signaling or cell proliferation in the presence of the HPR1 and/or HPR2 polypeptide and then determine if such intracellular signaling or cell proliferation is altered in the presence of a candidate agonist or antagonist or another HPR1 or HPR2 polypeptide. Exemplary assays for this aspect of the invention include cytokine secretion assays, cell proliferation assays, and mixed lymphocyte reactions involving antigen presenting cells and T cells. These assays are well known to those skilled in the art.

In another aspect, the present invention provides a method of detecting the ability of a test compound to affect the intracellular signaling or cell proliferation activity of a cell. In this aspect, the method comprises: (1) contacting a first group of target cells with a test compound including an HPR1 polypeptide and/or an HPR2 polypeptide, or a fragment or fragments thereof, under conditions appropriate to the particular assay being used; (2) measuring the net rate of intracellular signaling or cell proliferation among the target cells; and (3) observing the net rate of intracellular signaling or cell proliferation among control cells containing the HPR1 and./or HPR2 polypeptides or fragments thereof, in the absence of a test compound, under otherwise identical conditions as the first group of cells. In this embodiment, the net rate of intracellular signaling or cell proliferation in the control cells is compared to that of the cells treated with both a test compound and the HPR1 and/or HPR2 polypeptide(s). The comparison will provide a difference in the net rate of intracellular signaling or cell proliferation such that an effector of intracellular signaling or cell proliferation can be identified. The test compound can function as an effector by either activating or up-regulating, or by inhibiting or down-regulating, intracellular signaling or cell proliferation, and can be detected through this method.

Cell Proliferation. Cell Death, Cell Differentiation. and Cell Adhesion Assays. A polypeptide of the present invention may exhibit cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of a polypeptide of the present invention is evidenced by any one of a number of routine factor-dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK. The activity of an HPR1 or HPR2 polypeptide of the invention may, among other means, be measured by the following methods:

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7: Immunologic studies in humans); Takai et al, J. Immunol. 137: 3494-3500, 1986; Bertagnolli et al., J. Immunol. 145: 1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173: 1205-1211, 1991; Moreau et al., Nature 336:690-692, 1988; Greenberger et al., Proc Natl Acad Sci.USA 80: 2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83: 1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., Proc Natl Acad Sci USA 77: 6091-6095, 1980; Weinberger et al., Eur. J. Immun. 11:405-411, 1981; Takai et al., J. Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144: 3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., Proc Natl Acad Sci. USA 88:7548-7551, 1991

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, polypeptides that regulate lympho-hematopoiesis) include, without limitation, those described in: Methylcellulose colony forming assays, Freshney, 1994, In Culture of Hematopoietic Cells, Freshney et al. eds. pp. 265-268, Wiley-Liss, Inc., New York, N.Y.; Hirayama et al., Proc. Natl. Acad. Sci. USA 89:5907-5911, 1992; Primitive hematopoietic colony forming cells with high proliferative potential, McNiece and Briddell, 1994, In Culture of Hematopoietic Cells, Freshney et al. eds. pp. 23-39, Wiley-Liss, Inc., New York, N.Y.; Neben et al., Experimental Hematology 22:353-359, 1994; Ploemacher, 1994, Cobblestone area forming cell assay, In Culture of Hematopoietic Cells, Freshney et al. eds. pp. 1-21, Wiley-Liss, Inc., New York, N.Y.; Spooncer et al., 1994, Long term bone marrow cultures in the presence of stromal cells, In Culture of Hematopoietic Cells, Freshney et al. eds. pp. 163-179, Wiley-Liss, Inc., New York, N.Y.; Sutherland, 1994, Long term culture initiating cell assay, In Culture of Hematopoietic Cells, Freshney et al. eds. Vol pp. 139-162, Wiley-Liss, Inc., New York, N.Y.

Assays for tissue generation activity include, without limitation, those described in: International Patent Publication No. WO95116035 (bone, cartilage, tendon); International Patent Publication No. WO95/05846 (nerve, neuronal); International Patent Publication No. WO91/07491 (skin, endothelium). Assays for wound healing activity include, without limitation, those described in: Winter, Epidermal Wound Healing, pps. 71-112 (Maibach and Rovee, eds.), Year Book Medical Publishers, Inc., Chicago, as modified by Eaglstein and Mertz, J. Invest. Dermatol 71:382-84 (1978).

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Assays for cell movement and adhesion include, without limitation, those described in: Current Protocols in Immunology Coligan et al. eds, Greene Publishing Associates and Wiley-Interscience (Chapter 6.12, Measurement of alpha and beta chemokines 6.12.1-6.12.28); Taub et al. J. Clin. Invest. 95:1370-1376, 1995; Lind et al. APMIS 103:140-146, 1995; Muller et al Eur. J. Immunol. 25: 1744-1748; Gruber et al. J Immunol. 152:5860-5867, 1994; Johnston et al. J Immunol. 153: 1762-1768, 1994.

Assay for hemostatic and thrombolytic activity include, without limitation, those described in: Linet et al., J. Clin. Pharmacol. 26:131-140, 1986; Burdick et al., Thrombosis Res. 45:413-419,1987; Humphrey et al., Fibrinolysis 5:71-79 (1991); Schaub, Prostaglandins 35:467-474, 1988.

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63:1033-1038, 1990.

Diagnostic and Other Uses of HPR1 and HPR2 Polypeptides and Nucleic Acids

The nucleic acids encoding the HPR1 and HPR2 polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and. for gene therapy. Uses of HPR1 and HPR2 polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987

Probes and Primers. Among the uses of the disclosed HPR1 and HPR2 nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human HPR1 and HPR2 homologues.

Chromosome Mapping. The nucleic acids encoding HPR1 and HPR2 polypeptides, and the disclosed fragments and combinations of these nucleic acids, can be used by those skilled in the art using well-known techniques to identify the human chromosome to which these nucleic acids map. Useful techniques include, but are not limited to, using the sequence or portions, including oligonucleotides, as a probe in various well-known techniques such as radiation hybrid mapping (high resolution), in situ hybridization to chromosome spreads (moderate resolution), and Southern blot hybridization to hybrid cell lines containing individual human chromosomes (low resolution). Alternatively, the genomic sequences corresponding to nucleic acids encoding a cytokine polypeptide of the invention are mapped by comparison to sequences in public and proprietary databases, such as GenBank (ncbi.nlm.nih.gov/BLAST), Locuslink (ncbi.nlm.nih.gov:80/LocusLink/), Unigene (ncbi.nlm.nih.gov/cgi-bin/UniGene), AceView (ncbi.nlm.nih.gov/AceView), Gene Map Viewer (ncbi.nlm.nih.gov/genemap), Online Mendelian Inheritance in Man (OMIM) (ncbi.nlm.nih.gov/Omim), and proprietary databases such as the Celera Discovery System (celera.com). These computer analyses of available genomic sequence information can provide the identification of the specific chromosomal location of human and/or murine genomic sequences corresponding to sequences encoding HPR1 or HPR2 polypeptides of the invention, and the unique genetic mapping relationships between HPR1 or HPR2 genomic sequences and the genetic map locations of known human genetic disorders

Diagnostics and Gene Therapy. The nucleic acids encoding HPR1 and HPR2 polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The HPR1 and HPR2 polypeptides of the invention each can be used as reagents in methods to screen for or identify binding partners. For example, the HPR1 and HPR2 polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to Protein A- or Protein G-containing chromatography columns through interaction with the Fc moiety. The HPR1 and HPR2 polypeptides also find use in identifying cells that express a binding partner on the cell surface. Polypeptides are bound to a solid phase such as a column chromatography matrix or a similar suitable substrate. For example, magnetic microspheres can be coated with the polypeptides and held in an incubation vessel through a magnetic field. Suspensions of cell mixtures containing potential binding-partner-expressing cells are contacted with the solid phase having the polypeptides thereon. Cells expressing the binding partner on the cell surface bind to the fixed polypeptides, and unbound cells are washed away. Alternatively, HPR1 and HPR2 polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells to the beads. Procedures for using avidin-coated beads are known (see Berenson, et al. J. Cell Biochem., 10D:239, 1986). Washing to remove unbound material, and the release of the bound cells, are performed using conventional methods. In some instances, the above methods for screening for or identifying binding partners may also be used or modified to isolate or purify such binding partner molecules or cells expressing them.

Measuring Biological Activity. HPR1 and HPR2 polypeptides also find use in measuring the biological activity of HPR1-binding and/or HPR2-binding polypeptides in terms of their binding affinity. The polypeptides thus can be employed by those conducting “quality assurance” studies, e.g., to monitor shelf life and stability of polypeptide under different conditions. For example, the polypeptides can be employed in a binding affinity study to measure the biological activity of a binding partner polypeptide that has been stored at different temperatures, or produced in different cell types. The polypeptides also can be used to determine whether biological activity is retained after modification of a binding partner polypeptide (e.g., chemical modification, truncation, mutation, etc.). The binding affinity of the modified polypeptide is compared to that of an unmodified binding polypeptide to detect any adverse impact of the modifications on biological activity of the binding polypeptide. The biological activity of a binding polypeptide thus can be ascertained before it is used in a research study, for example.

Carriers and Delivery Agents. The polypeptides also find use as carriers for delivering agents attached thereto to cells bearing identified binding partners. The polypeptides thus can be used to deliver diagnostic or therapeutic agents to such cells (or to other cell types found to express binding partners on the cell surface) in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are ¹³¹I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent, for example. Conjugates comprising polypeptides and a suitable diagnostic or therapeutic agent (preferably covalently linked) are thus prepared. The conjugates are administered or otherwise employed in an amount appropriate for the particular application.

Treating Diseases Using HPR1 and/or HPR2 Polypeptides and Antagonists Thereof

It is anticipated that the HPR1 and HPR2 polypeptides, fragments, variants, antagonists, agonists, antibodies, and binding partners of the invention will be useful for treating medical conditions and diseases including, but not limited to, cell proliferation, metabolic, and reproductive hormone related conditions as described further herein. The therapeutic molecule or molecules to be used will depend on the etiology of the condition to be treated and the biological pathways involved, and variants, fragments, and binding partners of HPR1 and/or HPR2 polypeptides may have effects similar to or different from HPR1 or HPR2 polypeptides. For example, an antagonist of the ligand-binding activity of HPR1 and/or HPR2 polypeptides may be selected for treatment of conditions involving ligand-binding activity, but a particular fragment of a given HPR1 or HPR2 polypeptide may also act as an effective dominant negative antagonist of that activity. Therefore, in the following paragraphs “HPR1 and HPR2 polypeptides or antagonists” refers to all HPR1 and HPR2 polypeptides, fragments, variants, antagonists, agonists, antibodies, and binding partners etc. of the invention, and it is understood that a specific molecule or molecules can be selected from those provided as embodiments of the invention by individuals of skill in the art, according to the biological and therapeutic considerations described herein.

Also provided herein are methods for using HPR1 and HPR2 polypeptides or antagonists, compositions or combination therapies to treat various hematologic and oncologic disorders. For example, HPR1 and HPR2 polypeptides or antagonists are used to treat various forms of cancer, including acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia. Additional diseases treatable with the subject HPR1 and HPR2 polypeptides or antagonists, compositions or combination therapies are solid tumors, including sarcoma, osteosarcoma, and carcinoma, such as adenocarcinoma (for example, breast cancer) and squamous cell carcinoma. In addition, the subject compounds, compositions or combination therapies are useful for treating leukemia, including acute myelogenous leukemia, chronic or acute lymphoblastic leukemia and hairy cell leukemia. Other malignancies with invasive metastatic potential can be treated with the subject compounds, compositions and combination therapies, including multiple myeloma. In addition, the disclosed HPR1 and HPR2 polypeptides or antagonists, compositions and combination therapies can be used to treat anemias and hematologic disorders, including anemia of chronic disease, aplastic anemia, including Fanconi's aplastic anemia; idiopathic thrombocytopenic purpura (ITP); myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

Various lymphoproliferative disorders also are treatable with the disclosed HPR1 and HPR2 polypeptides or antagonists, compositions or combination therapies. These include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

In addition, the subject invention provides HPR1 and HPR2 polypeptides or antagonists, compositions and combination therapies for the treatment of non-arthritic medical conditions of the bones and joints. This encompasses osteoclast disorders that lead to bone loss, such as but not limited to osteoporosis, including post-menopausal osteoporosis, periodontitis resulting in tooth loosening or loss, and prosthesis loosening after joint replacement (generally associated with an inflammatory response to wear debris). This latter condition also is called “orthopedic implant osteolysis.” Another condition treatable by administering HPR1 and HPR2 polypeptides or antagonists, is temporal mandibular joint dysfunction (TMJ).

The disclosed HPR1 and HPR2 polypeptides or antagonists, compositions and combination therapies furthermore are useful for treating chronic neuronal degeneration.

Administration of HPR1 and HPR2 Polypeptides and Antagonists Thereof

This invention provides compounds, compositions, and methods for treating a patient, preferably a mammalian patient, and most preferably a human patient, who is suffering from a medical disorder, and in particular an HPR1- or HPR2-mediated disorder. Such HPR1- or HPR2-mediated disorders include conditions caused (directly or indirectly) or exacerbated by binding between HPR1 and/or HPR2 and a binding partner. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g., prophylactic) and palliative or ameliorative treatment. For such therapeutic uses, HPR1 and HPR2 polypeptides and fragments, HPR1 and HPR2 nucleic acids encoding the HPR1 and HPR2 polypeptides, and/or agonists or antagonists of the HPR1 and/or HPR2 polypeptides such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble HPR1 and/or HPR2 polypeptides.

Therapeutically Effective Amount. In practicing the method of treatment or use of the present invention, a therapeutically effective amount of a therapeutic agent of the present invention is administered to a patient having a condition to be treated, preferably to treat or ameliorate diseases associated with the activity of an HPR1 and/or HPR2 polypeptide. “Therapeutic agent” includes without limitation any of the HPR1 or HPR2 polypeptides, fragments, and variants; nucleic acids encoding the HPR1 and HPR2 polypeptides, fragments, and variants; agonists or antagonists of the HPR1 and HPR2 polypeptides such as antibodies; HPR1 and/or HPR2 polypeptide binding partners; complexes formed from the HPR1 and/or HPR2 polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as HPR1 and/or HPR2 polypeptides or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating acute conditions. For injuries or acute conditions, a single dose may be sufficient. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, HPR1 and/or HPR2 polypeptides or antagonists are administered one time per week to treat the various medical disorders disclosed herein, in another embodiment is administered at least two times per week, and in another embodiment is administered at least three times per week. If injected, the effective amount of HPR1 or HPR2 polypeptides or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing HPR1 and/or HPR2 polypeptides or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of HPR1 or HPR2 polypeptides or antagonists one to three times per week over a period of at least three weeks, or a dose of 50 mg of HPR1 or HPR2 polypeptides or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of HPR1 or HPR2 polypeptides or antagonists, administered by subcutaneous injection one or more times per week. If an antibody against an HPR1 and/or HPR2 polypeptide is used as the HPR1 and/or HPR2 polypeptide antagonist, a preferred dose range is 0.1 to 20 mg/kg, and more preferably is 1-10 mg/kg. Another preferred dose range for an anti-HPR1 polypeptide and/or anti-HPR2 polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of an HPR1 and/or HPR2 polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of HPR1 and/or HPR2 polypeptides are used. Sustained-release forms suitable for use in the disclosed methods include, but are not limited to, HPR1 and/or HPR2 polypeptides that are encapsulated in a slowly-dissolving biocompatible polymer (such as the alginate microparticles described in U.S. Pat. No. 6,036,978), admixed with such a polymer (including topically applied hydrogels), and or encased in a biocompatible semi-permeable implant.

Combinations of Therapeutic Compounds. An HPR1 or HPR2 polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of HPR1 and/or HPR2 polypeptides or antagonists concurrently with one or more other drugs that are administered to the same patient in combination with the HPR1 and/or HPR2 polypeptides or antagonists, each drug being administered according to a regimen suitable for that medicament. “Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be included in the pharmaceutical composition of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-11, IL-12, IL-13, IL-14, IL-15, IL-17, IL-18, IFN, TNF0, TNF1, TNF2, G-CSF, Meg-CSF, thrombopoietin, stem cell factor, and erythropoietin. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, an HPR1 and/or HPR2 polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, HPR1 and/or HPR2 polypeptides or antagonists can be combined with a second HPR1 and/or HPR2 polypeptide/antagonist, including an antibody against an HPR1 and/or HPR2 polypeptide, or an HPR1 polypeptide-derived peptide or HPR2 polypeptide-derived peptide that acts as a competitive inhibitor of native HPR1 and/or HPR2 polypeptides.

Routes of Administration. Any efficacious route of administration may be used to therapeutically administer HPR1 and HPR2 polypeptides or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation.; eyedrops; vaginal or rectal suppositories; buccal preparations; oral preparations, including pills, syrups, lozenges or chewing gum; and topical preparations such as lotions, gels, sprays, ointments or other suitable techniques. Alternatively, polypeptideaceous HPR1 and HPR2 polypeptides or antagonists may be administered by implanting cultured cells that express the polypeptide, for example, by implanting cells that express HPR1 and/or HPR2 polypeptides or antagonists. Cells may also be cultured ex vivo in the presence of polypeptides of the present invention in order to proliferate or to produce a desired effect on or activity in such cells. Treated cells can then be introduced in vivo for therapeutic purposes. In another embodiment, the patient's own cells are induced to produce HPR1 and/or HPR2 polypeptides or antagonists by transfection in vivo or ex vivo with a DNA that encodes HPR1 and/or HPR2 polypeptides or antagonists. This DNA can be introduced into the patient's cells, for example, by injecting naked DNA or liposome-encapsulated DNA that encodes HPR1 and/or HPR2 polypeptides or antagonists, or by other means of transfection. Nucleic acids of the invention can also be administered to patients by other known methods for introduction of nucleic acid into a cell or organism (including, without limitation, in the form of viral vectors or naked DNA). When HPR1 and/or HPR2 polypeptides or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition can desirably be encapsulated or injected in a viscous form for delivery to the site of bone, cartilage or tissue damage. Topical administration may be suitable for wound healing and tissue repair. Therapeutically useful agents other than a polypeptide of the invention which can also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides or extracellular matrix components. Other potential matrices are nonbiodegradable and chemically defined, such as sintered hydroxapatite, bioglass, aluminates, or other ceramics Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention can be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin-like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, HPR1 and HPR2 polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a TNFα-mediated inflammatory or arthritic condition. In such instances, an appropriate dose can be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. In a preferred embodiment, HPR1 and/or HPR2 polypeptides or antagonists (preferably constructed from genes derived from the same species as the patient), are administered by injection or other suitable route one or more times per week until the animal's condition is improved, or they can be administered indefinitely.

Manufacture of Medicaments. The present invention also relates to the use of HPR1 and HPR2 polypeptides, fragments, and variants; nucleic acids encoding the HPR1 or HPR2 polypeptides, fragments, and variants; agonists or antagonists of the HPR1 and/or HPR2 polypeptides such as antibodies; HPR1 and/or HPR2 polypeptide binding partners; complexes formed from the HPR1 and/or HPR2 polypeptides, fragments, variants, and binding partners, etc, in the manufacture of a medicament for the prevention or therapeutic treatment of each medical disorder disclosed herein.

### EXAMPLES

The following examples are intended to illustrate particular embodiments and not to limit the scope of the invention.

### Example 1

A. Identification of HPR1, a New Member of the Human Hematopoietin Receptor Family

A data set was received from Celera Genomics (Rockville, Md.) containing a listing of amino acid sequences predicted to be encoded by the human genome. This data set was searched with a BLAST algorithm to identify hematopoietin receptor family polypeptides. Several amino acid sequences, including two overlapping amino acid sequences (SEQ ID NO: 1 and SEQ ID NO: 2), were identified as comprising partial amino acid sequences of a new human hematopoietin receptor polypeptide, HPR1. These amino acids sequences were used to identify a DNA sequence (SEQ ID NO: 3) encoding an HPR1 polypeptide having the amino acid sequence shown in SEQ ID NO: 4; nucleotides 132 through 2366 of SEQ ID NO: 3 encode SEQ ID NO: 4, with nucleotides 2367 through 2369 corresponding to a stop codon. The HPR1 coding sequence (nucleotides 132 through 2369 of SEQ ID NO: 3) is presented as SEQ ID NO: 5. The HPR1 sequences of SEQ ID NOs 3 and 5 were confirmed by three independent PCR amplification experiments from a U937 cDNA library. These HPR1 coding sequences were compared with publicly available preliminary human genomic DNA sequences, and the following chromosome 5 contigs were identified as containing HPR1 coding sequences: AC022265.3, AC008914.3, AC008857.4, and AC016596.4. The human genomic region corresponding to these contigs also includes the gene for gp130, which suggests that gp130 and HPR1 may derive from a common ancestral gene by gene duplication. The approximate positions of the exons containing HPR1 coding sequence in the AC022265.3 contig are shown in the table below, along with their locations relative to SEQ ID NOs 3 and 5; note that the 5′ and 3′ untranslated regions may extend further along the contig sequence beyond those portions that correspond to SEQ ID NOs 3 and 5, as indicated by the parentheses around the AC022265.3 endpoints in the table. Due to the preliminary sequence and assembly of the contig sequence, the exons within the contig are not always in the right order or orientation with respect to each other, and may contain sequence variations due to inaccurate sequence data or allelic polymorphism.

Corresponding positions of HPR1 gene exons in human contig AC022265.3 and in cDNA sequences:

| | | Position in SEQ ID NO:3/ |
|---|---|---|
| | Position in AC022265.3 | Position in SEQ ID NO:5 |
| Exon 1 | (128423)-128559 | 1-137/1-6 |
| Exon 2 | 134501-134591 | 138-228/7-97 |
| Exon 3 | 143777-143894 | 229-346/98-215 |
| Exon 4 | 147256-147437 | 347-528/216-397 |
| Exon 5 | 51249-51098 | 529-680/398-549 |
| Exon 6 | 44322-44157 | 681-846/550-715 |
| Exon 7 | 16473-16394 | 847-926/716-795 |
| Exon 8 | 30331-30115 | 927-1143/796-1012 |
| Exon 9 | 178626-178808 | 1144-1326/1013-1195 |
| Exon 10 | 179879-179980 | 1327-1428/1196-1297 |
| Exon 11 | 180785-180931 | 1429-1575/1298-1444 |
| Exon 12 | 183052-183192 | 1576-1716/1445-1585 |
| Exon 13 | 185997-186090 | 1717-1810/1586-1679 |
| Exon 14 | 187367-187448 | 1811-1892/1680-1761 |
| Exon 15 | 189165-(189747) | 1893-2480/1762-2238 |

A nucleic acid encoding a polypeptide with a high degree of amino acid similarity (approximately 61% amino acid identity) to human HPR1 was isolated from Mus musculus. The Mus HPR1 amino acid sequence is presented as SEQ ID NO: 12, and due to its high level of similarity with human HPR1, is considered to be the murine homologue of human HPR1. PCR amplification of cDNA sequences corresponding to mRNAs encoding murine HPR1 identified a cDNA molecule encoding SEQ ID NO: 12; the nucleotide sequence of this murine HPR1 cDNA is presented as SEQ ID NO: 28. Nucleotides 1 through 2178 of SEQ ID NO: 28 encode SEQ ID NO: 12, with nucleotides 2179-2181 corresponding to a stop codon. Variants of the murine HPR1 amino acid sequence that are likely allelic variants have been identified in which the ‘T’ residue at position 121 of SEQ ID NO: 28 is changed to a ‘C’ residue, resulting in a change from the Phe residue at position 41 of SEQ ID NO: 4 to a Leu residue, and in which the ‘G’ residue at position 1666 of SEQ ID NO: 28 is changed to an ‘A’ residue, resulting in a change from the Asp residue at position 556 of SEQ ID NO: 4 to an Asn residue.

Several splice variations of the HPR1 sequences have been identified in human genomic sequences and are included within the scope of the invention. For example, amino acids 1 through 55 of SEQ ID NO: 1 match the amino acid sequence of HPR1 presented in SEQ ID NO: 4, while amino acids 56 through 77 of SEQ ID NO: 1 may be a portion of an alternatively spliced exon added following the exon/intron boundary identified between nucleotides 846 and 847 of SEQ ID NO: 3 (nucleotides 715 and 716 of SEQ ID NO: 5). In an additional potential splice variant, an amino acid sequence ending in the amino acids of SEQ ID NO: 10 could be substituted for the amino acids leading up to and including the lysine at position 190 of SEQ ID NO: 4. However, such a splice variant would require an additional exon/intron boundary approximately between nucleotides 701 and 702 of SEQ ID NO: 3 (nucleotides 570 and 571 of SEQ ID NO: 5). In a further potential splice variant, the amino acid sequence of SEQ ID NO: 11 could be substituted for amino acids 238 through 266 of SEQ ID NO: 4 by replacing exon 7 with an alternative exon encoding the SEQ ID NO: 11 amino acids. In this potential variant, 29 amino acids C-terminal to the WSXWS motif and including the N-terminal portion of the most N-terminal fibronectin type III repeat (as shown in Table 1) would be replaced with 15 amino acids, resulting in deletion of a portion of the most N-terminal fibronectin type III repeat, including two highly conserved Trp residues.

Additional variations of HPR1 polypeptides are provided as naturally occurring genomic variants of the HPR1 sequences disclosed herein; such variations may be incorporated into an HPR1 polypeptide or nucleic acid individually or in any combination, or in combination with alternative splice variation as described above. As one example, amino acids 5 through 40 of SEQ ID NO: 2 match SEQ ID NO: 4, with amino acid 4 of SEQ ID NO: 2 likely representing an allelic variation, where the change from the Asn residue position 187 of SEQ ID NO: 4 to a Thr residue in SEQ ID NO: 2 could be caused by a single change from ‘A’ to ‘C’ at position 691 of SEQ ID NO: 3 or 560 of SEQ ID NO: 5. This variation and others are listed in the table below:

| | Position in | | Position in SEQ ID NO:3/ |
|---|---|---|---|
| Amino Acid Change | SEQ ID NO:4 | Nucleotide Change | Position in SEQ ID NO:5 |
| Thr −> Ala | 83 | A −> G | 378/247 |
| Asp −> Asn | 168 | G −> A | 633/502 |
| Asn −> Thr | 187 | A −> C | 691/560 |
| Ser −> Pro | 361 | T −> C | 1212/1081 |
| Ala −> Gly | 362 | C −> G | 1216/1085 |
| Ser −> Asn | 510 | G −> A | 1660/1529 |
| Asn −> Asp | 517 | A −> G | 1680/1549 |
| Arg −> Gly | 679 | A −> G | 2166/2035 |

B. Identification of HPR2, a New Member of the Human Hematopoietin Receptor Family

A data set was received from Celera Genomics (Rockville, Md.) containing a listing of amino acid sequences predicted to be encoded by the human genome. This data set was searched with a BLAST algorithm to identify hematopoietin receptor family polypeptides. Several amino acid sequences, including SEQ ID NO: 16, were identified as comprising partial amino acid sequences of a new human hematopoietin receptor polypeptide, HPR2. These amino acids sequences were used to identify a DNA sequence (SEQ ID NO: 19) encoding an HPR2 polypeptide having the amino acid sequence shown in SEQ ID NO: 21; nucleotides 107 through 1993 of SEQ ID 19 encode SEQ ID NO: 21, with nucleotides 1994 through 1996 corresponding to a stop codon. The HPR2 coding sequence (nucleotides 107 through 1996 of SEQ ID NO: 19) is presented as SEQ ID NO: 20. The HPR2 sequences of SEQ ID NOs 19 and 20 were confirmed by independent PCR amplification experiments from a human lymph node cDNA library and a CB23 B cell line cDNA library. These PCR amplification experiments also identified two additional splice variants of the HPR2 cDNA sequence referred to as HPR2-ex8-ex9 and HPR2-ex9; the coding sequences for HPR2-ex8-ex9 and HPR2-are presented as SEQ ID NOs 22 and 24, respectively, and the amino acid sequences they encode are presented as SEQ ID NOs 23 and 25, respectively. The HPR2 cDNA sequences of SEQ ID NOs 19, 20, and the HPR2-ex8-ex9 cDNA of SEQ ID NO: 22 were present in both the lymph node and CB23 cDNA libraries, while the HPR2-ex9 cDNA of SEQ ID NO: 24 was only present in the lymph node library.

These HPR2 coding sequences were compared with publicly available preliminary human genomic DNA sequences, and the following chromosome 1 contigs were identified as containing HPR2 coding sequences: GenBank accession numbers AL109843 (1p31.2-32.1) and AL389925. The human genomic region corresponding to the AL389925 contig also includes the gene for IL-12RB2, which suggests that IL-12RB2 and HPR2 may derive from a common ancestral gene by gene duplication. The approximate positions of the exons containing HPR2 coding sequence in the AL109843 and AL389925 contigs are shown in the table below, along with their locations relative to SEQ ID NOs 19, 20, 22, and 24; note that the 5′ and 3′ untranslated regions may extend further along the contig sequence beyond those portions that correspond to SEQ ID NOs 19, 20, 22, and 24, as indicated by the parentheses around the AL109843 and AL389925 endpoints in the table. Due to the preliminary nature of the sequence data and assembly of the contig sequence, the exons within the genomic contigs may contain sequence variations due to inaccurate sequence data or allelic polymorphism.

Corresponding positions of HPR2 gene exons in human genomic contigs AL109843 and AL389925 and in HPR2 coding sequences:

| | | Position in SEQ ID NO:19/20/22/24 |
|---|---|---|
| | Position in AL109843 | |
| Exon 1 | (34088)-34164 | 1-77/(5' UTR, not in SEQ ID NOs 20, 22, and 24) |
| Exon 2 | 35715-35813 | 78-176/1-70/1-70/1-70 |
| Exon 3 | 36965-37261 | 177-473/71-367/71-367/71-367 |
| Exon 4 | 50459-50582 | 474-597/368-491/368-491/368-491 |
| Exon 5 | 68360-68520 | 598-758/492-652/492-652/492-652 |
| Exon 6 | 74533-74678 | 759-904/653-798/653-798/653-798 |
| Exon 7 | 87197-87353 | 905-1061/799-955/799-955/799-955 |
| Exon 8 | 104336-104425 | 1062-1151/956-1045/(not resent)/956-1045 |
| Exon 9 | 107802-107904 | 1152-1254/1046-1148/(not present)/(not present) |
| | Position in AL389925 | |
| Exon 10 | 8847-8937 | 1255-1345/1149-1239/‘G’-957-1047/1046-1071 |
| Exon 11 | 11488-(12972) | 1346-2830/1240-1890/1048-1698/(not present) |

In the HPR3-ex9 splice variant, note that the absence of the exon 9 sequence (103 nucleotides) changes the reading frame towards the 3′ end of the coding sequence for the HPR2-ex9 form (SEQ ID NO: 24) relative to that of the HPR2 coding sequence of SEQ ID NO: 20, leading to a different amino acid sequence in the HPR2-ex9 C-terminal portion and a stop codon after amino acid 356 (compared to 629 amino acids in HPR2). For the HPR2-ex8-ex9 form, the splice is made at a slightly different exon 10 splice acceptor site than for the HPR2 form, so that an extra ‘G’ residue is included at the start of exon 10 in the HPR2-ex8-ex9 form, restoring the reading frame to be the same as in the 3′ end of the HPR2 sequence. The C-terminal 248 amino acids of HPR2-ex8-ex9 form are therefore the same as the C-terminal 248 amino acids of HPR2 form, and although the coding sequence of the HPR2-ex8-ex9 form is missing both exons 8 and 9 (except for the last ‘G’ residue of exon 9), the resulting HPR2-ex8-ex9 form polypeptide is longer (565 amino acids) than the HPR2-ex9 form polypeptide (356 amino acids).

Several splice variations of the HPR2 sequences have been identified in human genomic sequences and are included within the scope of the invention. For example, amino acids 118 through 215 of SEQ ID NO: 16 match the amino acid sequence of HPR2 presented in SEQ ID NO: 21, while amino acids 1 through 117 of SEQ ID NO: 16 may correspond to an alternatively spliced exon added upstream of exon 3 (i.e. at the exon/intron boundary identified between nucleotides 176 and 177 of SEQ ID NO: 19). Amino acids 216 through 245 of SEQ ID NO: 16 may correspond to an additional alternatively spliced exon added between exon 3 and exon 4 (i.e. at the exon/intron boundary identified between nucleotides 473 and 474 of SEQ ID NO: 19). Amino acids 340 through 344 of SEQ ID NO: 16 may correspond to an alternatively spliced exon added downstream of exon 5 (i.e. at the exon/intron boundary identified between nucleotides 758 and 759 of SEQ ID NO: 19). In a further potential splice variant, an alternative exon or exons encoding the amino acid sequence of SEQ ID NO: 17 could be substituted for exon 6, resulting in the replacement of amino acids 217 through 267 of SEQ ID NO: 21 with the SEQ ID NO: 17 amino acids. In this potential variant, 51 amino acids N-terminal to the WSXWS motif, including the proline-rich region (as shown in Table 1) between the two cytokine receptor subdomains, would be replaced with 39 amino acids, resulting in deletion of a portion of the more C-terminal cytokine receptor subdomain which includes a highly conserved Trp residue. In an additional potential splice variant, an alternative exon could be added downstream of exon 4 (i.e. at the exon/intron boundary identified between nucleotides 597 and 598 of SEQ ID NO: 19) so that an amino acid sequence starting in the amino acids of SEQ ID NO: 18 could be substituted for amino acids following and including the serine at position 164 of SEQ ID NO: 21. Multiple splice variations as described above can be included in a single splice variant, for example, replacing exon 6 with an alternative exon or exons encoding the amino acid sequence of SEQ ID NO: 17, and also deleting exons 8 and/or 9 as described above.

Additional variations of HPR2 polypeptides are provided as naturally occurring genomic variants of the HPR2 sequences disclosed herein; such variations may be incorporated into an HPR2 polypeptide or nucleic acid individually or in any combination, or in combination with alternative splice variation as described above. As one example, a change from the Leu residue position 310 of SEQ ID NO: 21 to a Pro residue could be caused by a single change from ‘T’ to ‘C’ at position 1035 of SEQ ID NO: 19. This variation and another are listed in the table below:

| | Position in | Nucleotide | Position in |
|---|---|---|---|
| Amino Acid Change | SEQ ID NO:21 | Change | SEQ ID NO:19 |
| Leu −> Pro | 310 | T −> C | 1035 |
| (not applicable) | (not applicable) | A −> G | 2172 (3' UTR) |

A nucleic acid encoding a polypeptide with a high degree of amino acid similarity (approximately 69% amino acid identity) to human HPR2 was isolated from Mus musculus. The Mus HPR2 amino acid sequence is presented as SEQ ID NO: 27, and due to its high level of similarity with human HPR2, is considered to be the murine homologue of human HPR2. PCR amplification of cDNA sequences corresponding to mRNAs encoding murine HPR2 identified a cDNA molecule encoding SEQ ID NO: 27; the nucleotide sequence of this murine HPR2 cDNA is presented as SEQ ID NO: 29. Nucleotides 1 through 1932 of SEQ ID NO: 29 encode SEQ ID NO: 27, with nucleotides 1933-1935 corresponding to a stop codon. The murine HPR2 amino acid sequence of SEQ ID NO: 27 appears to have a 20-amino acid insertion at amino acids 297 through 316 of SEQ ID NO: 27 relative to human HPR2 of SEQ ID NO: 21, based on an alignment of the human and murine polypeptide sequences; this insertion is identical to amino acids 317 through 336. Given the number of alternatively spliced forms identified for human HPR2, it is possible that this insertion in murine HPR2 relative to the human HPR2 of SEQ ID NO: 21 is the result of alternative splicing. One embodiment of the invention is a form of murine HPR2 in which one of these repeated WQPWS—containing motifs has been deleted; that is, polypeptides in which the amino acid sequence ending with amino acid 296 of SEQ ID NO: 27 is contiguous with the amino acid sequence beginning with amino acid 317 of SEQ ID NO: 27, or polypeptides in which the amino acid sequence ending with amino acid 316 of SEQ ID NO: 27 is contiguous with the amino acid sequence beginning with amino acid 337 of SEQ ID NO: 27.

C. Comparison of HPR1 and HPR2 to Other Hematopoietin Receptor Polypeptides.

The amino acid sequences of human HPR1 (SEQ ID NO: 4), murine HPR1 (SEQ ID NO: 12), and human HPR2 (SEQ ID NO: 21) were compared with the amino acid sequences of these other hematopoietin receptor family members—LIF-R, the interleukin 12 beta 2 receptor chain (IL-12RB2), gp130, and GCSFR (SEQ ID NO: 6-SEQ ID NO: 9, respectively)—using the GCG “pretty” multiple sequence alignment program, with amino acid similarity scoring matrix=blosum62, gap creation penalty=8, and gap extension penalty=2. Alignments of these sequences are shown in Table 1, and include consensus residues which are identical among at least three of the amino acid sequences in the alignment. The capitalized residues in the alignment are those which match the consensus residues. The numbering of amino acid residues in Table 1 corresponds to the position of those residues in the HPR1 amino acid sequence (SEQ ID NO: 4). Note that only a portion of the HPR2 amino acid sequence is shown in Table 1, as HPR2 does not contain fibronectin type III repeats in its extracellular domain. HPR1 and HPR2 sequences corresponding to the intracellular Box 1 and Box 2 motifs are shown in Table 2. Sequences of eleven amino acids similar to the Box 1 or 2 motif of other hematopoietin receptors were identified for HPR1 and HPR2, and placed into a column with these motif sequences (with no gaps introduced). Similarly, HPR2 sequences corresponding to the intracellular Box 3 motif are shown in Table 3. Sequences of fourteen amino acids similar to the Box 3 motif of other hematopoietin receptors were identified for HPR2, and placed into a column with these motif sequences (with no gaps introduced). The numbering of each sequence on Tables 2 and 3 corresponds to their position in the complete amino acid sequence for that HPR polypeptide. The consensus residues are those that are present in three or more (for Table 2) or two or more (for Table 3) sequences at that position in the motif.

Amino acid substitutions and other alterations (deletions, insertions, etc.) to HPR1 and HPR2 amino acid sequences (for example, SEQ ID NOs 4, 12, and 21) are predicted to be more likely to alter or disrupt HPR1 or HPR2 polypeptide activities if they result in changes to the capitalized residues of the amino acid sequences as shown in Tables 1, 2, and 3, and particularly if those changes do not substitute an amino acid of similar structure (such as substitution of any one of the aliphatic residues-Ala, Gly, Leu, Ile, or Val—for another aliphatic residue), or a residue present in other hematopoietin receptor polypeptides at that conserved position. Conversely, if a change is made to an HPR1 or HPR2 amino acid sequence resulting in substitution of the residue at that position in the alignment from one of the other Table 1, 2, or 3 hematopoietin receptor polypeptide sequences, it is less likely that such an alteration will affect the function of the altered HPR1 or HPR2 polypeptide. For example, the consensus residue at position 42 in Table 1 is serine, and one of the hematopoietin receptors (LIF-R) has an asparagine at that position. Substitution of asparagine or the chemically similar glutamine for serine at that position is considered to be less likely to alter the function of the polypeptide than substitution of tryptophan or tyrosine etc. Embodiments of the invention include HPR1 and HPR2 polypeptides and fragments of HPR1 and HPR2 polypeptides, comprising altered amino acid sequences. Altered HPR1 or HPR2 polypeptide sequences share at least 30%, or more preferably at least 40%, or more preferably at least 50%, or more preferably at least 55%, or more preferably at least 60%, or more preferably at least 65%, or more preferably at least 70%, or more preferably at least 75%, or more preferably at least 80%, or more preferably at least 85%, or more preferably at least 90%, or more preferably at least 95%, or more preferably at least 97.5%, or more preferably at least 99%, or most preferably at least 99.5% amino acid identity with one or more of the hematopoietin receptor amino acid sequences shown in Tables 1, 2, and 3.

TABLE 1
Alignment of HPR1 and HPR2 extracellular domains with those of other hematopoietin receptors

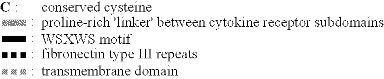

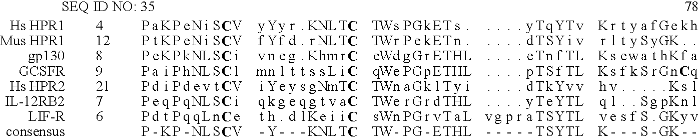

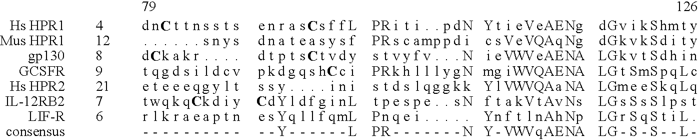

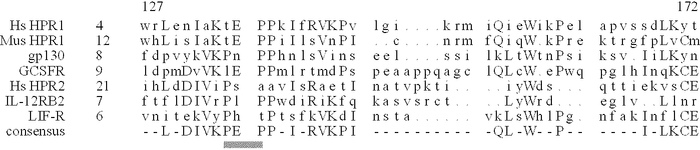

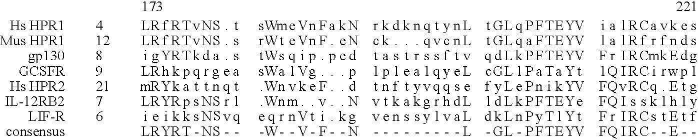

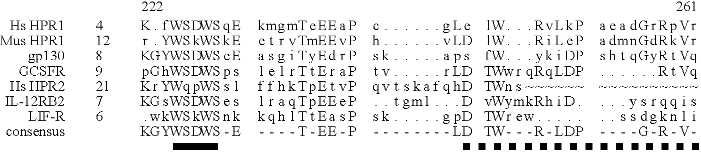

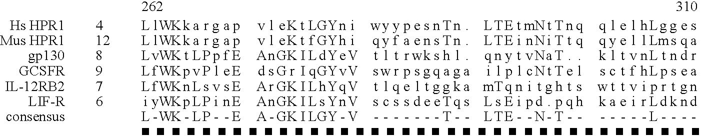

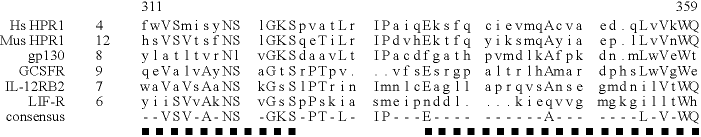

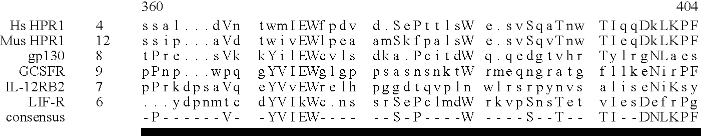

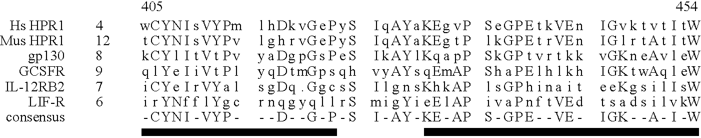

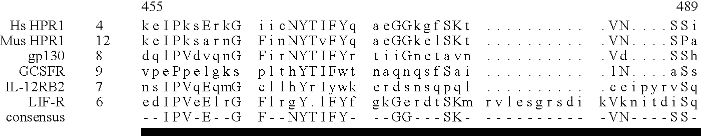

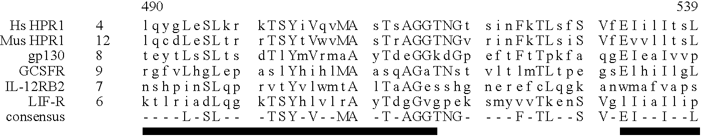

TABLE 2
Box 1 and Box 2 motifs in the intracellular domains of
HPR1, HPR2, and other hematopoietin receptors

| | SEQ ID NO | Box 1 Motif | Box 2 Motif |
|---|---|---|---|
| Hs HPR1 | 4 | 563-thlcWPtVPNP-573 | 631-eifTdEArtgq-641 |
| Mus HPR1 | 12 | 517-tplccPDVPNP-527 | 582-Vv1TEEAgKgq-592 |
| HPR2 | 21 | 393-pkwlyeDiPNm-403 | 430-VdpmiteiKei-440 |
| LIF-R | 6 | 866-KetfyPDiPNP-876 | 910-VleTrsAfpKi-920 |
| gp130 | 8 | 648-KkhiWPnVPdP-658 | 693-VveiEandKKp-703 |
| GCSFR | 9 | 655-KnplWPsVPdP-665 | 696-ltvlEEdeKKp-706 |
| consensus | | K---WPDVPNP | V--TEEA-KK- |

TABLE 3
Box 3 motifs in the intracellular domains of HPR2 and
other hematopoietin receptors

| | SEQ ID NO | Box 3 Motif |
|---|---|---|
| HPR2 (first occurrence) | 21 | 478-PdLntGYKPQisnf-491 |
| HPR2 (second occurrence) | 21 | 605-lpsintYfPQniLe-618 |
| LIF-R | 6 | 995-PVggaGYKPQmhLp-1008 |
| gp130 | 8 | 693-tVvhsGYrhQvpsv-774 |
| GCSFR | 9 | 696-PtLvqtYvlQgdpr-734 |
| consensus residues | | PVL--GYKPQ--L- |

### EXAMPLE 2

### Monoclonal Antibodies That Bind Polypeptides of the Invention

This example illustrates a method for preparing monoclonal antibodies that bind HPR1 or HPR2 polypeptides. Suitable immunogens that may be employed in generating such antibodies include, but are not limited to, purified HPR1 or HPR2 polypeptide or an immunogenic fragment thereof.

Purified HPR1 or HPR2 polypeptide can be used to generate monoclonal antibodies immunoreactive therewith, using conventional techniques such as those described in U.S. Pat. No. 4,411,993. Briefly, mice are immunized with HPR1 or HPR2 polypeptide immunogen emulsified in complete Freund's adjuvant, and injected in amounts ranging from about 10 to about 100 micrograms subcutaneously or intraperitoneally. Ten to twelve days later, the immunized animals are boosted with additional HPR1 or HPR2 polypeptide emulsified in incomplete Freund's adjuvant. Mice are periodically boosted thereafter on a weekly to bi-weekly immunization schedule. Serum samples are periodically taken by retro-orbital bleeding or tail-tip excision to test for anti-HPR1 or anti-HPR2 antibodies by dot blot assay, ELISA (Enzyme-Linked Immunosorbent Assay), or inhibition of binding of HPR1 or HPR2 polypeptide to an HPR1 and/or HPR2 binding partner.

Following detection of an appropriate antibody titer, positive animals are provided one last intravenous injection of HPR1 or HPR2 polypeptide in saline. Three to four days later, the animals are sacrificed, spleen cells harvested, and spleen cells are fused to a murine myeloma cell line, e.g., NS1 or preferably P3x63Ag8.653 (ATCC CRL 1580). Fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells are screened by ELISA for reactivity against purified HPR1 or HPR2 polypeptide by adaptations of the techniques disclosed in Engvall et al., (Immunochem. 8:871, 1971) and in U.S. Pat. No. 4,703,004. A preferred screening technique is the antibody capture technique described in Beckmann et al., (J. Immunol. 144:4212, 1990). Positive hybridoma cells can be injected intraperitoneally into syngeneic BALB/c mice to produce ascites containing high concentrations of anti-HPR1 or anti-HPR2 monoclonal antibodies. Alternatively, hybridoma cells can be grown in vitro in flasks or roller bottles by various techniques. Monoclonal antibodies produced in mouse ascites can be purified by ammonium sulfate precipitation, followed by gel exclusion chromatography. Alternatively, affinity chromatography based upon binding of antibody to Polypeptide A or Polypeptide G can also be used, as can affinity chromatography based upon binding to HPR1 or HPR2 polypeptide.

### EXAMPLE 3

### Antisense Inhibition of HPR1 and/or HPR2 Nucleic Acid Expression

In accordance with the present invention, a series of oligonucleotides are designed to target different regions of HPR1 and/or HPR2 human or murine mRNA molecules, using the nucleotide sequences of SEQ ID NOs 3, 5, 19, 20, 22, 24, 28, and 29 as the bases for the design of the oligonucleotides. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyldiphenyl-silyl-5-methyluridine, 5′-0-tert-butyldiphenylsilyl-2′-0-[(2-formadoximinooxy)ethyl]-5-methyluridine, 5′-O-tert-butyldiphenylsilyl-2′-O-[N,N-dimethylaminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropylphosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxytrityl)guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]—[2′-deoxy]—[2′-O-Me] chimeric phosphorothioate oligonucleotides, [2′-O-(2-methoxyethyl)]—[2′-deoxy]—[2′-O-(methoxyethyl)] chimeric phosphorothioate oligonucleotides, and [2′-O-(2-methoxy-ethyl)phosphodiester]—[2′-deoxy phosphoro-thioate]—[2′-O-(2-methoxyethyl) phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleotides/oligonucleosides are synthesized according to U.S. Pat. No. 5,623,065.

Oligonucleotides are preferably synthesized via solid phase P(III) phosphoramidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of HPR1 and/or HPR2 nucleic acid expression can be assayed in a variety of ways known in the art. For example, HPR1 and HPR2 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is =accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. HPR1 and HPR2 protein levels can be quantitated in a variety of ways well known in the art, such as immunoprecipitation, Western blot analysis (immunoblotting), ELISA, or fluorescence-activated cell sorting (FACS). Antibodies directed to HPR1 and/or HPR2 polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated polypeptide having HPR1 polypeptide activity comprising an amino acid sequence selected from the group consisting of:
(a) the amino acid sequence of SEQ ID NO: 4;
(b) an amino acid sequence selected from the group consisting of: amino acids 652 though 745 of SEQ ID NO: 4, a fragment of the sequence of amino acids 652 though 745 of SEQ ID NO: 4 comprising at least 20 contiguous amino acids; a fragment of the sequence of amino acids 652 though 745 of SEQ ID NO: 4 comprising at least 30 contiguous amino acids; a fragment of the sequence of amino acids 652 though 745 of SEQ ID NO: 4 that is at least 25% of the length of the sequence of amino acids 652 though 745 of SEQ ID NO: 4; a fragment of the sequence of amino acids 652 though 745 of SEQ ID NO: 4 that is at least 50% of the length of the sequence of amino acids 652 though 745 of SEQ ID NO: 4; and a fragment of the sequence of amino acids 652 though 745 of SEQ ID NO: 4 comprising at least eight contiguous amino acids and comprising at least one tyrosine residue;
(c) an amino acid sequence comprising at least 8 amino acids and sharing amino acid identity with the amino acid sequences of (b), wherein the percent amino acid identity is selected from the group consisting of: at least 70%, at least 75%, at least 80%, at least 85%, at least 90%, at least 95%, at least 97.5%, at least 99%, and at least 99.5%;
(d) an amino acid sequence comprising both an amino acid sequence of (b) or (c), and an amino acid sequence selected from the group consisting of: amino acids 1 through 55 of SEQ ID NO: 1; amino acids 56 through 77 of SEQ ID NO: 1; amino acids 5 through 40 of SEQ ID NO: 2; amino acids 1 through 32 of SEQ ID NO: 4; amino acids 1 through 241 of SEQ ID NO: 4; amino acids 1 through 525 of SEQ ID NO: 4; amino acids 20 through 32 of SEQ ID NO: 4; amino acids 33 through 134 of SEQ ID NO: 4; amino acids Xaa1 through Xaa2 of SEQ ID NO: 4, wherein Xaa1 is selected from the group consisting of amino acids 33 through 43 of SEQ ID NO: 4 and Xaa2 is selected from the group consisting of amino acids 228 through 241 of SEQ ID NO: 4; amino acids 33 through 238 of SEQ ID NO: 4; amino acids 33 through 241 of SEQ ID NO: 4; amino acids 33 through 525 of SEQ ID NO: 4; amino acids 33 through 745 of SEQ ID NO: 4; amino acids 44 through 94 of SEQ ID NO: 4; amino acids 139 through 241 of SEQ ID NO: 4; amino acids 242 through 326 of SEQ ID NO: 4; amino acids 242 through 514 of SEQ ID NO: 4; amino acids 337 through 419 of SEQ ID NO: 4; amino acids 433 through 514 of SEQ ID NO: 4; amino acids 526 through 556 of SEQ ID NO: 4; amino acids 533 through 552 of SEQ ID NO: 4; amino acids 553 through 745 of SEQ ID NO: 4; amino acids 557 through 745 of SEQ ID NO: 4; amino acids 563 through 573 of SEQ ID NO: 4; amino acids 563 through 641 of SEQ ID NO: 4; amino acids 567 through 581 of SEQ ID NO: 4; amino acids 588 through 639 of SEQ ID NO: 4; amino acids 631 through 641 of SEQ ID NO: 4; SEQ ID NO: 10; and SEQ ID NO: 11;
(e) an amino acid sequence comprising both an amino acid sequence of (b) or (c), and a fragment of SEQ ID NO: 4 comprising cytokine receptor domain amino acid sequences;
(f) an allelic variant of any of (a)-(e); and
(g) an amino acid sequence of (a)-(f), wherein a polypeptide comprising said amino acid sequence of (a)-(f) binds to an antibody that also binds to a polypeptide comprising an amino acid sequence of any of (b)-(c).

2. An isolated polypeptide having HPR1 polypeptide activity comprising an amino acid sequence selected from the group consisting of:
(a) the amino acid sequence of SEQ ID NO: 12;
(b) an amino acid sequence selected from the group consisting of: amino acids 633 though 726 of SEQ ID NO: 12, a fragment of the sequence of amino acids 633 though 726 of SEQ ID NO: 12 comprising at least 20 contiguous amino acids; a fragment of the sequence of amino acids 633 though 726 of SEQ ID NO: 12 comprising at least 30 contiguous amino acids; a fragment of the sequence of amino acids 633 though 726 of SEQ ID NO: 12 that is at least 25% of the length of the sequence of amino acids 633 though 726 of SEQ ID NO: 12; a fragment of the sequence of amino acids 633 though 726 of SEQ ID NO: 12 that is at least 50% of the length of the sequence of amino acids 633 though 726 of SEQ ID NO: 12; and a fragment of the sequence of amino acids 633 though 726 of SEQ ID NO: 12 comprising at least eight contiguous amino acids and comprising at least one tyrosine residue;
(c) an amino acid sequence comprising at least 8 amino acids and sharing amino acid identity with the amino acid sequences of (b), wherein the percent amino acid identity is selected from the group consisting of: at least 70%, at least 75%, at least 80%, at least 85%, at least 90%, at least 95%, at least 97.5%, at least 99%, and at least 99.5%;
(d) an amino acid sequence comprising both an amino acid sequence of (b) or (c), and an amino acid sequence selected from the group consisting of: amino acids 1 through 28 of SEQ ID NO: 12; amino acids 1 through 224 of SEQ ID NO: 12; amino acids 1 through 509 of SEQ ID NO: 12; amino acids 13 through 28 of SEQ ID NO: 12; amino acids 29 through 124 of SEQ ID NO: 12; amino acids Xaa1 through Xaa2 of SEQ ID NO: 12, wherein Xaa1 is selected from the group consisting of amino acids 29 through 39 of SEQ ID NO: 12 and Xaa2 is selected from the group consisting of amino acids 211 through 224 of SEQ ID NO: 12; amino acids 29 through 128 of SEQ ID NO: 12; amino acids 29 through 224 of SEQ ID NO: 12; amino acids 29 through 509 of SEQ ID NO: 12; amino acids 29 through 726 of SEQ ID NO: 12; amino acids 129 through 224 of SEQ ID NO: 12; amino acids 225 through 309 of SEQ ID NO: 12; amino acids 225 through 499 of SEQ ID NO: 12;
(e) an amino acid sequence comprising both an amino acid sequence of (b) or (c), and a fragment of SEQ ID NO: 12 comprising cytokine receptor domain amino acid sequences;
(f) an allelic variant of any of (a)-(e); and
(g) an amino acid sequence of (a)-(f), wherein a polypeptide comprising said amino acid sequence of (a)-(f) binds to an antibody that also binds to a polypeptide comprising an amino acid sequence of any of (b)-(c).

3. An isolated polypeptide having HPR2 polypeptide activity comprising an amino acid sequence selected from the group consisting of:
(a) SEQ ID NO: 23;
(b) SEQ ID NO: 25;
(c) an amino acid sequence selected from the group consisting of: an amino acid sequence comprising at least 20 contiguous amino acids of SEQ ID NO: 23 and comprising the contiguous amino acids 318 and 319 of SEQ ID NO: 23; and amino acids 349 through 356 of SEQ ID NO: 25;
(d) an amino acid sequence comprising at least 8 amino acids and sharing amino acid identity with the amino acid sequences of (c), wherein the percent amino acid identity is selected from the group consisting of: at least 70%, at least 75%, at least 80%, at least 85%, at least 90%, at least 95%, at least 97.5%, at least 99%, and at least 99.5%;
(e) an amino acid sequence comprising both an amino acid sequence of (c) or (d), and an amino acid sequence selected from the group consisting of: amino acids 1 through 177 of SEQ ID NO: 16; amino acids 216 through 245 of SEQ ID NO: 16; SEQ ID NO: 17; and SEQ ID NO: 18;
(f) an amino acid sequence comprising both an amino acid sequence of (c) or (d), and an amino acid sequences of any of (a)-(b) comprising cytokine receptor domain amino acid sequences;
(g) an allelic variant of any of (a)-(f); and
(h) an amino acid sequence of (a)-(g), wherein a polypeptide comprising said amino acid sequence of (a)-(g) binds to an antibody that also binds to a polypeptide comprising an amino acid sequence of any of (c)-(d).

4. An isolated polypeptide having HPR2 polypeptide activity comprising an amino acid sequence selected from the group consisting of:
(a) SEQ ID NO: 27;
(b) SEQ ID NO: 27 from which amino acids 297 through 316 or amino acids 317 through 336 have been deleted;
(c) an amino acid sequence comprising 20 or more contiguous amino acids of (a) or (b); and
(d) an amino acid sequence comprising 30 or more contiguous amino and sharing at least 90% amino acid identity with the amino acid sequences of (a)-(b).

5. An isolated polypeptide having HPR1 polypeptide activity comprising an amino acid sequence selected from the group consisting of:
(a) the amino acid sequence of SEQ ID NO: 4;
(b) an amino acid sequence selected from the group consisting of: amino acids 652 though 745 of SEQ ID NO: 4, a fragment of the sequence of amino acids 652 though 745 of SEQ ID NO: 4 comprising at least 20 contiguous amino acids; a fragment of the sequence of amino acids 652 though 745 of SEQ ID NO: 4 comprising at least 30 contiguous amino acids; a fragment of the sequence of amino acids 652 though 745 of SEQ ID NO: 4 that is at least 25% of the length of the sequence of amino acids 652 though 745 of SEQ ID NO: 4; a fragment of the sequence of amino acids 652 though 745 of SEQ ID NO: 4 that is at least 50% of the length of the sequence of amino acids 652 though 745 of SEQ ID NO: 4; and a fragment of the sequence of amino acids 652 though 745 of SEQ ID NO: 4 comprising at least eight contiguous amino acids and comprising at least one tyrosine residue;
(c) an amino acid sequence comprising at least 8 amino acids and sharing amino acid identity with the amino acid sequences of (b), wherein the percent amino acid identity is selected from the group consisting of: at least 80%, at least 85%, at least 90%, at least 95%, at least 97.5%, at least 99%, and at least 99.5%; and
(d) an amino acid sequence comprising both an amino acid sequence of (b) or (c), and an amino acid sequence selected from the group consisting of: amino acids 1 through 55 of SEQ ID NO: 1; amino acids 56 through 77 of SEQ ID NO: 1; amino acids 5 through 40 of SEQ ID NO: 2; amino acids 1 through 32 of SEQ ID NO: 4; amino acids 1 through 241 of SEQ ID NO: 4; amino acids 1 through 525 of SEQ ID NO: 4; amino acids 20 through 32 of SEQ ID NO: 4; amino acids 33 through 134 of SEQ ID NO: 4; amino acids Xaa1 through Xaa2 of SEQ ID NO: 4, wherein Xaa1 is selected from the group consisting of amino acids 33 through 43 of SEQ ID NO: 4 and Xaa2 is selected from the group consisting of amino acids 228 through 241 of SEQ ID NO: 4; amino acids 33 through 238 of SEQ ID NO: 4; amino acids 33 through 241 of SEQ ID NO: 4; amino acids 33 through 525 of SEQ ID NO: 4; amino acids 33 through 745 of SEQ ID NO: 4; amino acids 44 through 94 of SEQ ID NO: 4; amino acids 139 through 241 of SEQ ID NO: 4; amino acids 242 through 326 of SEQ ID NO: 4; amino acids 242 through 514 of SEQ ID NO: 4; amino acids 337 through 419 of SEQ ID NO: 4; amino acids 433 through 514 of SEQ ID NO: 4; amino acids 526 through 556 of SEQ ID NO: 4; amino acids 533 through 552 of SEQ ID NO: 4; amino acids 553 through 745 of SEQ ID NO: 4; amino acids 557 through 745 of SEQ ID NO: 4; amino acids 563 through 573 of SEQ ID NO: 4; amino acids 563 through 641 of SEQ ID NO: 4; amino acids 567 through 581 of SEQ ID NO: 4; amino acids 588 through 639 of SEQ ID NO: 4; amino acids 631 through 641 of SEQ ID NO: 4; SEQ ID NO: 10; and SEQ ID NO: 11.

6. An isolated polypeptide having HPR1 polypeptide activity comprising an amino acid sequence selected from the group consisting of:
(a) the amino acid sequence of SEQ ID NO: 12;
(b) an amino acid sequence selected from the group consisting of: amino acids 633 though 726 of SEQ ID NO: 12, a fragment of the sequence of amino acids 633 though 726 of SEQ ID NO: 12 comprising at least 20 contiguous amino acids; a fragment of the sequence of amino acids 633 though 726 of SEQ ID NO: 12 comprising at least 30 contiguous amino acids; a fragment of the sequence of amino acids 633 though 726 of SEQ ID NO: 12 that is at least 25% of the length of the sequence of amino acids 633 though 726 of SEQ ID NO: 12; a fragment of the sequence of amino acids 633 though 726 of SEQ ID NO: 12 that is at least 50% of the length of the sequence of amino acids 633 though 726 of SEQ ID NO: 12; and a fragment of the sequence of amino acids 633 though 726 of SEQ ID NO: 12 comprising at least eight contiguous amino acids and comprising at least one tyrosine residue;
(c) an amino acid sequence comprising at least 8 amino acids and sharing amino acid identity with the amino acid sequences of (b), wherein the percent amino acid identity is selected from the group consisting of: at least 80%, at least 85%, at least 90%, at least 95%, at least 97.5%, at least 99%, and at least 99.5%; and
(d) an amino acid sequence comprising both an amino acid sequence of (b) or (c), and an amino acid sequence selected from the group consisting of: amino acids 1 through 28 of SEQ ID NO: 12; amino acids 1 through 224 of SEQ ID NO: 12; amino acids 1 through 509 of SEQ ID NO: 12; amino acids 13 through 28 of SEQ ID NO: 12; amino acids 29 through 124 of SEQ ID NO: 12; amino acids Xaa1 through Xaa2 of SEQ ID NO: 12, wherein Xaa1 is selected from the group consisting of amino acids 29 through 39 of SEQ ID NO: 12 and Xaa2 is selected from the group consisting of amino acids 211 through 224 of SEQ ID NO: 12; amino acids 29 through 128 of SEQ ID NO: 12; amino acids 29 through 224 of SEQ ID NO: 12; amino acids 29 through 509 of SEQ ID NO: 12; amino acids 29 through 726 of SEQ ID NO: 12; amino acids 129 through 224 of SEQ ID NO: 12; amino acids 225 through 309 of SEQ ID NO: 12; amino acids 225 through 499 of SEQ ID NO: 12.

7. An isolated polypeptide having HPR2 polypeptide activity comprising an amino acid sequence selected from the group consisting of:
(a) SEQ ID NO: 23;
(b) SEQ ID NO: 25;
(c) an amino acid sequence selected from the group consisting of: an amino acid sequence comprising at least 20 contiguous amino acids of SEQ ID NO: 23 and comprising the contiguous amino acids 318 and 319 of SEQ ID NO: 23; and amino acids 349 through 356 of SEQ ID NO: 25;
(d) an amino acid sequence comprising at least 8 amino acids and sharing amino acid identity with the amino acid sequences of (c), wherein the percent amino acid identity is selected from the group consisting of: at least 80%, at least 85%, at least 90%, at least 95%, at least 97.5%, at least 99%, and at least 99.5%; and
(e) an amino acid sequence comprising both an amino acid sequence of (c) or (d), and an amino acid sequence selected from the group consisting of: amino acids 1 through 177 of SEQ ID NO: 16; amino acids 216 through 245 of SEQ ID NO: 16; SEQ ID NO: 17; and SEQ ID NO: 18.

8. An isolated polypeptide having HPR2 polypeptide activity comprising an amino acid sequence selected from the group consisting of:
(a) SEQ ID NO: 27;
(b) SEQ ID NO: 27 from which amino acids 297 through 316 or amino acids 317 through 336 have been deleted; and
(c) an amino acid sequence comprising 30 or more contiguous amino acids of (a) or (b).

9. A method for identifying compounds that alter HPR1 polypeptide activity comprising
(a) contacting the polypeptide of claim 1 with a test compound; and
(b) determining whether the test compound alters the effect on intracellular signaling of said polypeptide.

10. A method for identifying compounds that alter HPR1 polypeptide activity comprising
(a) contacting the polypeptide of claim 2 with a test compound; and
(b) determining whether the test compound alters the effect on intracellular signaling of said polypeptide.

11. A method for identifying compounds that alter HPR2 polypeptide activity comprising
(a) contacting the polypeptide of claim 3 with a test compound; and
(b) determining whether the test compound alters the effect on intracellular signaling of said polypeptide.

12. A method for identifying compounds that alter HPR2 polypeptide activity comprising
(a) contacting the polypeptide of claim 4 with a test compound; and
(b) determining whether the test compound alters the effect on intracellular signaling of said polypeptide.
